(19) United States
(12) Patent Application Publication
(10) Pub. No.: US 2026/0288822 A1
(43) Pub. Date: Sep. 24, 2026

(54) SYSTEM

(71) Applicant: SoftBank Group Corp., Tokyo (JP)

(72) Inventor: Jiahe ZOU, Tokyo (JP)

(73) Assignee: SoftBank Group Corp., Tokyo (JP)

(21) Appl. No.: 19/568,813

(22) Filed: Mar. 17, 2026

(30) Foreign Application Priority Data
Mar. 19, 2025 (JP) 2025-045175

(51) Int. Cl. G06F 16/3329; G10L 25/63

(52) U.S. Cl. CPC G06F 16/33295; G10L 25/63

(57) ABSTRACT

A system includes a processor that is configured to receive, as a prompt to a generative AI model, an input from an animal owner and initiate a dialog with the animal owner based on the prompt, control acquisition of data regarding a health condition and a behavior pattern of an animal by using at least one of a sensor and a camera, and perform preprocessing and analysis on the acquired data, and analyze an emotional state of the animal owner by using an emotion recognition technique and adjust a response of the generative AI model based on a result of the analysis of the emotional state.

### CROSS-REFERENCE TO RELATED APPLICATION

This application is based on and claims priority under 35 USC 119 from Japanese Patent Application No. 2025-045175 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional animal care support systems that utilize sensors, cameras, or simple rule-based engines generally focus on objective data such as health indicators and behavior patterns of an animal. However, such systems do not adequately consider the emotional state of the animal owner during interaction with the system. As a result, even when accurate analytical information about the animal is provided, the provided advice or answers may be perceived as insensitive, overly technical, or insufficiently supportive, thereby reducing user satisfaction and adherence to recommended care or training plans.

Furthermore, in systems that merely incorporate a generative AI model for dialog, the response style is typically uniform and does not dynamically adapt to fluctuations in the owner's emotional state, such as anxiety, confusion, or distress. This lack of emotional adaptation can lead to misunderstandings, diminished trust in the system, and suboptimal outcomes in animal health management, training, and nutrition management. Additionally, existing systems often fail to tightly integrate sensor-based analysis of the animal with emotion-aware dialog generation, so that advice, training plans, and nutrition support are not optimized in a holistic manner.

Accordingly, there is a need for a system that integrates: (i) data collection and analysis of an animal's health condition and behavior pattern using sensors and cameras, (ii) dialog generation using a generative AI model, and (iii) emotion recognition of the animal owner, such that the system can adjust and optimize responses, advice, training plans, and nutrition management support in real time according to both the objective state of the animal and the subjective emotional state of the owner.

### SUMMARY

In order to solve the above-described problems, the present invention provides a system comprising a processor, wherein the processor is configured to receive, as a prompt to a generative AI model, an input from an animal owner and to initiate a dialog with the animal owner based on the prompt. The processor is further configured to control acquisition of data regarding a health condition and a behavior pattern of an animal by using at least one of a sensor and a camera, and to perform preprocessing and analysis on the acquired data. In addition, the processor is configured to analyze an emotional state of the animal owner by using an emotion recognition technique and to adjust a response of the generative AI model based on a result of the analysis of the emotional state.

According to one aspect of the invention, the processor is configured to automatically generate an answer to a question from the animal owner and to adjust the answer based on the emotion recognition technique. For example, when the emotion recognition technique detects a high level of anxiety or distress in the owner, the processor can cause the generative AI model to produce responses that are more reassuring, explanatory, and stepwise, thereby improving the owner's understanding and comfort.

According to another aspect of the invention, the processor is configured to automatically adjust at least one of advice, a training plan, and nutrition management support for the animal owner based on an analysis result of the health condition and the behavior pattern of the animal, and to optimize the adjustment based on the emotion recognition technique. In particular, the processor can adapt the depth, tone, and granularity of the recommendations according to the detected emotional state of the owner, and can prioritize or defer certain types of instructions depending on whether the owner appears calm, confused, or overwhelmed. By combining sensor-and camera-based analysis of the animal with emotion-aware dialog generation, the system can provide personalized, context-sensitive support that enhances user satisfaction and promotes more effective implementation of animal care, training, and nutrition management.

The term “system” refers to an integrated combination of hardware and software components configured to perform data acquisition, analysis, and dialog generation functions as described in the claims.

The term “processor” refers to one or more hardware processing units, such as a central processing unit (CPU), a graphics processing unit (GPU), or a dedicated accelerator, which execute instructions to perform the predetermined functions of the system.

The term “generative AI model” refers to a machine learning model, such as a large language model or other neural network-based model, that is trained to generate natural-language or other content in response to an input prompt.

The term “prompt” refers to input data, including natural-language text or structured information, provided to the generative AI model to condition or guide the content of a generated response.

The term “animal owner” refers to a human user who keeps, manages, or cares for an animal and who interacts with the system by providing inputs and receiving outputs.

The term “dialog” refers to an interactive exchange of information between the system and the animal owner, including sequences of questions, answers, advice, and confirmations generated and processed over time.

The term “sensor” refers to any device capable of detecting and outputting data related to a physical quantity associated with the animal, such as motion, temperature, heart rate, location, or environmental conditions.

The term “camera” refers to an image-capturing device configured to obtain still images or video of the animal, which can be used to derive information about the animal's health condition or behavior pattern.

The term “health condition” refers to a physical or physiological state of the animal, including but not limited to signs of illness, injury, stress, fatigue, or other medical or wellness-related states, as inferable from sensor, camera, or other data.

The term “behavior pattern” refers to a temporal sequence or statistical trend of observable actions or activities of the animal, such as movement, rest, play, feeding, or other behavioral indicators, derived from sensor data, camera data, or user input.

The term “data acquisition” refers to the process of obtaining, receiving, and recording data related to the animal's health condition or behavior pattern from at least one of a sensor and a camera.

The term “preprocessing” refers to operations applied to raw acquired data, including but not limited to cleaning, filtering, normalization, feature extraction, and formatting, to make the data suitable for analysis.

The term “analysis” refers to computational processing and evaluation of preprocessed data for the purpose of deriving metrics, patterns, classifications, anomalies, or other information regarding the animal's health condition or behavior pattern.

The term “emotion recognition technique” refers to a method or algorithm, including but not limited to machine learning-based or rule-based methods, for estimating or classifying an emotional state of the animal owner from input such as voice, text, facial images, or interaction history.

The term “emotional state” refers to a psychological or affective condition of the animal owner, such as anxiety, calmness, distress, confusion, or satisfaction, as determined or estimated by the emotion recognition technique.

The term “response of the generative AI model” refers to output content, such as natural-language text or other generated information, produced by the generative AI model in reaction to a prompt or sequence of prompts.

The term “answer” refers to a generated response that is provided by the system to address a specific question or inquiry from the animal owner.

The term “advice” refers to guidance or recommendations generated by the system regarding care, management, observation, or handling of the animal, based on at least one of the animal's health condition, behavior pattern, or the owner's emotional state.

The term “training plan” refers to a structured set of instructions, schedules, or strategies generated by the system for teaching, conditioning, or modifying the behavior of the animal.

The term “nutrition management support” refers to guidance, planning, or recommendations generated by the system concerning the type, amount, timing, or method of feeding the animal and managing its dietary intake.

The term “adjust” refers to the act of modifying at least one of the content, style, tone, detail level, or timing of a response, answer, advice, training plan, or nutrition management support generated by the system based on one or more inputs, including the emotional state of the animal owner.

The term “optimize the adjustment” refers to refining or selecting an adjustment in a manner that aims to improve the relevance, comprehensibility, acceptance, or effectiveness of the system's outputs with respect to both the animal's state and the animal owner's emotional state.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

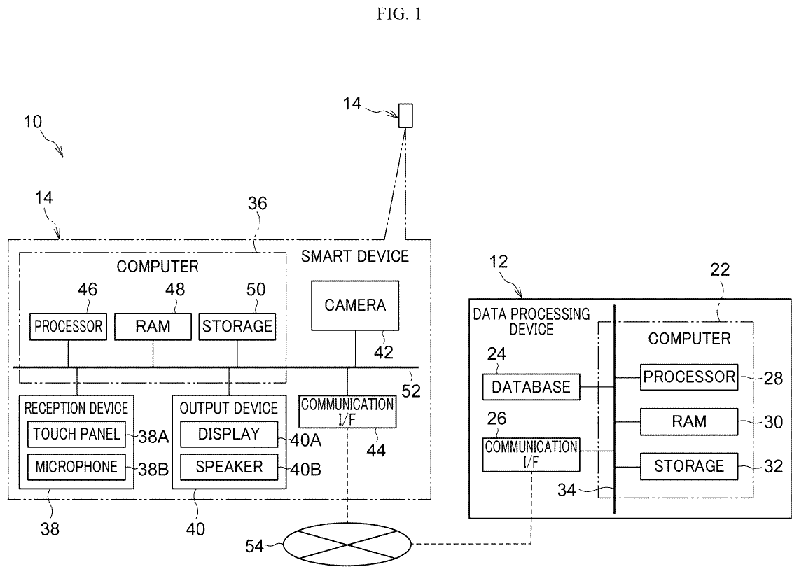

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

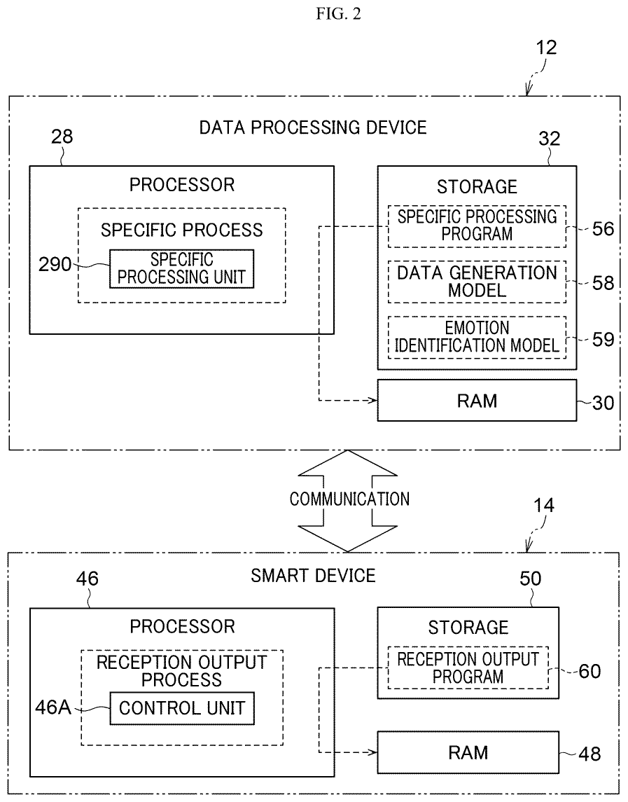

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional animal-care support systems that utilize conversational artificial intelligence models often treat each user inquiry as an isolated text input and generate a generic response solely based on the immediate inquiry content. Such systems typically lack mechanisms to systematically structure the user's input as a machine-optimized prompt, to incorporate longitudinal animal-specific data stored in backend resources, or to persist prior interactions as reusable context. As a result, the generated responses are frequently oversimplified, inconsistent across sessions, and insufficiently tailored to the individual animal's health status and behavior history.

From a computer-technology perspective, known architectures generally implement a straightforward client-server pipeline in which a terminal merely forwards raw text to a remote model endpoint and displays the model's output. In these configurations, the server does not perform coordinated control over (i) standardized formatting of prompts, (ii) integration of heterogeneous data sources such as health records and behavior logs, and (iii) iterative reuse of interaction history to refine subsequent model inputs. This leads to inefficient utilization of computational resources in the server and storage subsystems, and to suboptimal behavior of the generative model, which cannot exploit available structured data and history for improved inference.

Furthermore, existing systems rarely define, at the processor level, explicit processing stages that manage the transformation of user input into a structured prompt sentence, the controlled invocation of a generative information processing model, and the post-processing of the model's response by combining it with data retrieved from storage. Without such well-defined processing stages, it is difficult to guarantee reproducibility, scalability, and maintainability of the system behavior, and it is challenging to ensure that the same hardware and software resources can consistently deliver high-quality, context-aware responses.

Accordingly, there is a need for a computer-implemented system that improves the way a processor coordinates a terminal, an information processing apparatus, a storage device, and a generative information processing model. The system should standardize prompt generation at the terminal side, orchestrate model invocation and data retrieval at the server side, and maintain interaction history in a form directly usable as subsequent model input. By improving these internal computer operations, the system can enhance response relevance and consistency while more effectively exploiting available computational and storage resources.

The specific processing by the specific processing unit 290 of the data processing device 12 in Example 1 is realized by the following means.

The present invention provides a server comprising a processor configured to receive, from a terminal, a prompt sentence constituting inquiry information to be input to a generative information processing model, to generate communication information including the prompt sentence, and to transmit the communication information to an information processing apparatus; to extract, in the information processing apparatus, the prompt sentence from the communication information, to generate analysis information including the prompt sentence and history information related to an animal, to input the analysis information to the generative information processing model, and to cause the generative information processing model to generate response information based on natural language processing; to access, in the information processing apparatus, a storage device storing health information and behavior information of the animal, and to generate personalized response information by adding supplementary information based on the health information and the behavior information to the response information; to transmit, from the information processing apparatus, the personalized response information to the terminal and to cause the terminal to convert the personalized response information into display information for presentation via an output device; and to record, as history information, the prompt sentence and the personalized response information transmitted between the terminal and the information processing apparatus and to make the history information available as subsequent input information to the generative information processing model. This enables a technically improved interaction pipeline in which the processor standardizes and enriches prompts before model invocation, programmatically fuses model outputs with structured historical data, and persistently manages interaction history for reuse, thereby enhancing the effectiveness, efficiency, and consistency of computer-implemented animal-care support.

The term “system” refers to an arrangement of one or more hardware and software components, including at least a processor, a terminal, an information processing apparatus, and a storage device, that cooperate to execute the claimed processing.

The term “processor” refers to one or more hardware-based computation units, such as a central processing unit or a processing core, configured by software or firmware to execute instructions for performing the claimed functions.

The term “terminal” refers to an information input and output apparatus operated by a user, including at least an input interface and a display or other output interface, and configured to transmit and receive data to and from an information processing apparatus over a communication network.

The term “information processing apparatus” refers to a computing apparatus, distinct from or including the terminal, that executes server-side processing such as reception of communication information, interaction with a generative information processing model, data retrieval from a storage device, and generation of personalized response information.

The term “storage device” refers to a memory resource, such as a magnetic storage, semiconductor memory, or other non-transitory computer-readable medium, configured to store health information, behavior information, and history information related to an animal and user interactions.

The term “prompt sentence” refers to text data that represents inquiry information or instructions to be provided as input to a generative information processing model in order to cause the model to generate response information.

The term “inquiry information” refers to information representing a request, question, or issue provided by a caretaker or other user regarding an animal's health status, behavior, or care, which is to be processed by the system.

The term “communication information” refers to structured data including at least a prompt sentence and optionally additional metadata, formatted for transmission between the terminal and the information processing apparatus.

The term “analysis information” refers to data provided to a generative information processing model, including at least a prompt sentence and history information related to an animal, used by the model to generate response information.

The term “generative information processing model” refers to a machine learning model, such as a generative artificial intelligence model, configured to receive text or other input data and to generate response information through natural language processing or similar generative processing.

The term “response information” refers to output data generated by the generative information processing model based on analysis information, the output data including at least one natural-language expression addressing the inquiry information.

The term “personalized response information” refers to response information that has been modified or supplemented by the information processing apparatus using health information, behavior information, or other stored data relating to a specific animal so that the response is tailored to that animal.

The term “health information” refers to data representing physical or mental conditions of an animal, including at least one of body weight, appetite, activity level, medical history, or other measurable health-related parameters.

The term “behavior information” refers to data representing behavior patterns or actions of an animal, including at least one of play behavior, sleep patterns, feeding behavior, or other observable activities.

The term “history information” refers to data representing past interactions, records, or events relating to an animal and a user, including at least previously transmitted prompt sentences, previously generated personalized response information, and past health information and behavior information.

The term “supplementary information” refers to additional data derived from health information, behavior information, or history information, such as statistics, trends, or contextual annotations, that is added to response information to generate personalized response information.

The term “display information” refers to data formatted by the terminal for presentation to a user through an output device, such as a display screen, audio output, or other user interface.

The term “output device” refers to a component of the terminal, such as a display, speaker, or haptic actuator, configured to present display information to a user.

The term “caretaker” refers to an individual or organization responsible for managing or supervising an animal and providing inquiry information regarding the animal to the system.

The term “animal attribute information” refers to data indicating characteristics of an animal, including at least one of species, breed, age, sex, size, or other identifying properties.

The term “caregiving situation information” refers to data describing the environment or conditions under which the animal is kept, including at least housing conditions, feeding schedule, exercise pattern, or other contextual caregiving details.

The term “standardized prompt sentence” refers to a prompt sentence that has been automatically formatted according to a predetermined structure or template suitable for input to a generative information processing model.

The term “structured inquiry information” refers to inquiry information arranged in a predefined data structure, including at least a standardized prompt sentence and one or more associated metadata fields, enabling efficient processing by the information processing apparatus and the generative information processing model.

The term “support information” refers to information generated based on response information and stored data, including at least exercise plan information, behavior training plan information, and nutrition management plan information for an animal.

The term “exercise plan information” refers to information describing recommended physical activities for an animal, including at least suggested duration, frequency, or type of exercise.

The term “behavior training plan information” refers to information describing recommended training actions to modify or support an animal's behavior, including at least step-by-step instructions, schedules, or reinforcement strategies.

The term “nutrition management plan information” refers to information describing recommended feeding-related actions for an animal, including at least suggested types of food, feeding amounts, feeding frequency, or nutrient balance.

The term “natural language processing” refers to computational techniques performed by a generative information processing model to interpret, generate, or transform human language text based on learned parameters.

In an embodiment, the server, the terminal, and the user cooperate to implement a computer-implemented animal-care support system that uses a generative AI model. The server uses one or more processors, a main memory, a non-transitory storage device, and a network interface mounted in a computing apparatus such as a rack-mounted computer or a virtual machine in a data center. The terminal uses a processor, a memory, a display, an input interface, and a wireless or wired communication module mounted in an information terminal such as a smartphone, a tablet, or a general-purpose computer. The user operates the terminal to input inquiry information regarding an animal.

The terminal executes an application program implemented, for example, using a mobile application framework. The terminal stores the application program in a non-transitory storage device such as flash memory and loads the program into a random access memory when the user launches the application. The terminal then executes instructions of the program by means of the terminal processor.

The server executes a server-side application implemented, for example, using a web application framework running on an operating system such as a general-purpose server operating system. The server stores the application and configuration files in a magnetic storage device or a solid-state drive and loads execution modules into the main memory. The server further accesses a database management system, for example a relational database engine, that manages tables storing health information and behavior information related to multiple animals.

The server, in one embodiment, integrates a generative AI model in the form of a neural network model deployed on an accelerator such as a graphics processing unit. The generative AI model is implemented as a transformer-based sequence model including an embedding layer, multiple self-attention layers, feed-forward layers, and an output projection layer. The server stores trained parameters of the model, including weight matrices and bias vectors, in a model storage area and loads them into the accelerator memory at inference time. The server configures the model to accept a prompt sentence as tokenized input and to output a sequence of tokens representing response information.

The terminal, in one example, allows the user to input a question regarding an animal such as:

“My 4-year-old dog has been sleeping much more than usual and is eating a bit less. What should I do?”

The terminal displays an input screen and receives touch or keyboard events through its input interface. The terminal converts these events into character data and stores the character data as a text string in the terminal memory. The terminal then standardizes the format of the text string according to a predetermined template. The terminal, for example, generates a prompt sentence such as:

“The user has a 4-year-old dog. The dog has been sleeping more than usual and has a slightly reduced appetite over the last week. As a generative AI model, analyze possible causes related to health and behavior, and provide clear, step-by-step recommendations, including when to consult a veterinarian.”

The terminal, in addition, may attach structured metadata that is not directly visible to the user but is maintained in the terminal memory, for example, species=dog, age=4, sex=male, and housing type=indoor. The terminal constructs an internal data structure that contains the standardized prompt sentence and the metadata. The terminal then converts the internal data structure into a representation suitable for transmission via a communication protocol. The terminal uses a communication stack implemented in the operating system, including a transport layer module and a network layer module, and transmits the data to the server over a wireless communication path such as a cellular network or a wireless local area network.

The server receives the transmitted data by means of its network interface and copies the received bytes into a receive buffer in the main memory. The server parses headers and payload and reconstructs the original communication information including the prompt sentence and the associated metadata. The server then constructs analysis information by combining the prompt sentence with history information retrieved from the database. The server, for example, retrieves a record of past weight values, activity durations, and prior inquiries related to the same animal from the database. The server stores the retrieved records in structured arrays or objects in the main memory.

The server generates a token sequence from the prompt sentence using a tokenizer module associated with the generative AI model. The tokenizer module maps substrings of the prompt sentence to integer token identifiers according to a vocabulary table. The server then constructs a model input tensor from the token identifiers and from positional encoding values and forwards the input tensor to the generative AI model running on the accelerator.

The generative AI model, in this embodiment, uses a transformer architecture. The model applies self-attention mechanisms across the token sequence to compute attention scores and weighted sums of token embeddings. The model calculates intermediate representations in multiple layers, each layer comprising multi-head attention sublayers and position-wise feed-forward sublayers. The model uses learned weight matrices that have been optimized during a pre-training stage on a large corpus of text data and optionally fine-tuned on domain-specific animal-care data.

The server configures the model to minimize a cross-entropy loss during training, using backpropagation and gradient-based optimization such as the Adam optimizer. During training, the model receives sequences of tokens and target next tokens, computes prediction distributions, and updates its weight parameters by computing gradients of the loss function with respect to the parameters. The server may apply regularization techniques such as dropout to reduce overfitting and data augmentation techniques such as synonym replacement to increase robustness. By performing these learning operations, the server configures the generative AI model to capture complex patterns in animal-related text and to generate coherent and context-sensitive responses.

The server, during inference in operation, performs a forward pass through the trained model only, without updating weights. The server obtains probability distributions over possible next tokens and selects tokens according to a decoding strategy such as greedy decoding or beam search with temperature control. This decoding process yields a sequence of token identifiers which the server converts back to characters to obtain a response text.

The server, after obtaining the initial response text, merges the response text with the health information and behavior information stored in the database. The server calculates statistical summaries such as average daily exercise time, weight change percentages over specified intervals, or frequency of specific symptoms noted in past inquiries. The server encodes these statistics into natural language segments and inserts them into appropriate positions in the response text according to predetermined rules. For example, if a stored weight trend exceeds a threshold, the server inserts a sentence such as “According to past records, your dog's weight has increased by 2 kg in the last three months, which may indicate a risk of overweight.”

The server uses rule-based logic tables that specify conditions on the statistics and the history information and associate the conditions with corresponding textual templates or emphasis levels. This rule-based post-processing enables the server to adjust the strength and urgency of recommendations based on quantitative data. The server then forms personalized response information, which is stored as a text string and optionally as a structured object including fields for exercise plan information, behavior training plan information, and nutrition management plan information.

The server sends the personalized response information back to the terminal using the network interface, and the terminal receives and stores the information in a display buffer.

The terminal converts the response text into display objects such as text views or list items and renders them on the display. The terminal may highlight specific phrases associated with urgency or risk using different colors or fonts. The user reads the displayed recommendations and may adjust animal-care routines accordingly.

In another example, the user inputs an inquiry such as:

“A 3-year-old indoor cat has started scratching furniture more often and meowing at night.

Please analyze possible behavioral causes and propose a step-by-step training plan to reduce these behaviors, including environmental enrichment ideas.”

The terminal transforms this inquiry into a prompt sentence compliant with the template and includes cat-specific attributes and environmental information. The server combines the user's prompt sentence with stored behavior information such as previously recorded scratching frequency or sleeping times and passes the combined information to the generative AI model. The generative AI model, based on its trained attention weights and sequence modeling capability, generates detailed behavioral guidance that is subsequently refined by the server using stored behavior logs and rule-based templates. The combination of neural network inference and structured data post-processing enables the server to produce more precise and context-aware training plans than would be obtained by using the generative AI model alone.

From a computer-technology standpoint, the server improves internal data management and processing efficiency by standardizing the structure of prompt sentences and by defining explicit internal data structures for communication information, analysis information, response information, and personalized response information. The server, by organizing data into these distinct categories, reduces the overhead associated with repeated parsing and formatting operations. The server can cache tokenized versions of frequently used template segments or metadata descriptions in memory and re-use them across multiple inferences, reducing the number of tokenization operations and thereby decreasing total computation time.

The server further improves accuracy and stability of generated responses by systematically injecting historical context and quantitative statistics into the model's effective input. In contrast to simple systems that only pass single-turn user text to a generative AI model, the described system maintains and revisits persistent history information, including past prompt sentences and past personalized responses, stored in the database. The server aggregates such history information into summarized descriptors used as additional context tokens in the prompt sentence. This aggregation offers a technically specific mechanism for leveraging long-term user-animal interactions without exceeding token length limits of the model, thereby reducing truncation-related errors and improving the continuity of advice.

The server also reduces communication load by shifting certain standardization tasks to the terminal. The terminal transforms free-form input into standardized prompt sentences and attaches structured metadata before transmission. This configuration allows the server to receive already normalized data, thereby reducing the need for complex natural language parsing on the server side and lowering the size of transmitted data because the terminal can reuse compact attribute codes. As a result, overall bandwidth consumption and server-side pre-processing time are reduced.

The generative AI model executes internal numerical operations that differ substantially from human reasoning processes. The model represents text as high-dimensional vectors and processes these vectors through matrix multiplications, non-linear activation functions, and attention weight calculations. The server uses these operations to identify statistical correlations and hidden patterns in large corpora that are not easily accessible to rule-based or manual methods. The attention mechanism, in particular, allows the model to assign varying importance to different parts of the prompt sentence and to different pieces of historical context, thus generating more focused outputs. The combination of these numerical methods with structured database queries yields an overall system that provides highly tailored outputs while optimizing hardware utilization of processors, memory, and accelerators.

The server can, in other embodiments, use different neural network architectures or different training regimes. For example, the server can use an encoder-decoder architecture instead of a decoder-only transformer, or can apply fine-tuning using supervised data from veterinary experts with domain-specific loss functions that penalize medically unsafe recommendations more heavily. The server may also implement reinforcement learning from human feedback, wherein human reviewers rate system outputs and the model updates its policy parameters to favor safer and more useful responses. These variations allow the system to adapt to different technical requirements while maintaining the same underlying data flow architecture.

The terminal, in alternative embodiments, can be implemented as a dedicated animal-care device that includes environmental sensors or wearable devices attached to the animal. In such cases, the terminal collects raw sensor values such as activity counts, temperature, or sound levels and packages them as part of the metadata accompanying the prompt sentence.

The server then incorporates this additional sensor information into the analysis information and uses it to produce richer and more precise responses. This configuration ties the abstract text processing of the generative AI model to physical measurements in the real world, thereby extending the system's technical effect beyond purely abstract data manipulation.

The user, across these embodiments, does not need to manually manage complex logs or compute statistical metrics. The server automatically maintains the history information in structured form in the storage device and links new inquiries with corresponding animal identifiers. This automated linkage and summarization process, which the processor carries out systematically and repeatedly, reduces the likelihood of human error in data handling and ensures that relevant historical context is always available to enhance new inferences. This leads to reduced response variance, improved prediction stability, and more reliable care recommendations from the computational standpoint.

In summary, the server, the terminal, and the user cooperate in a system where the processor performs specific and detailed data transformations: standardized prompt sentence generation, tokenization, neural-network-based generative computation, structured database querying and aggregation, and rule-based post-processing. These operations, implemented as concrete algorithmic steps on particular hardware resources, improve processing speed, response quality, data management efficiency, and communication efficiency, thereby providing a technical improvement in computer-based animal-care support beyond mere automation of human advice-giving.

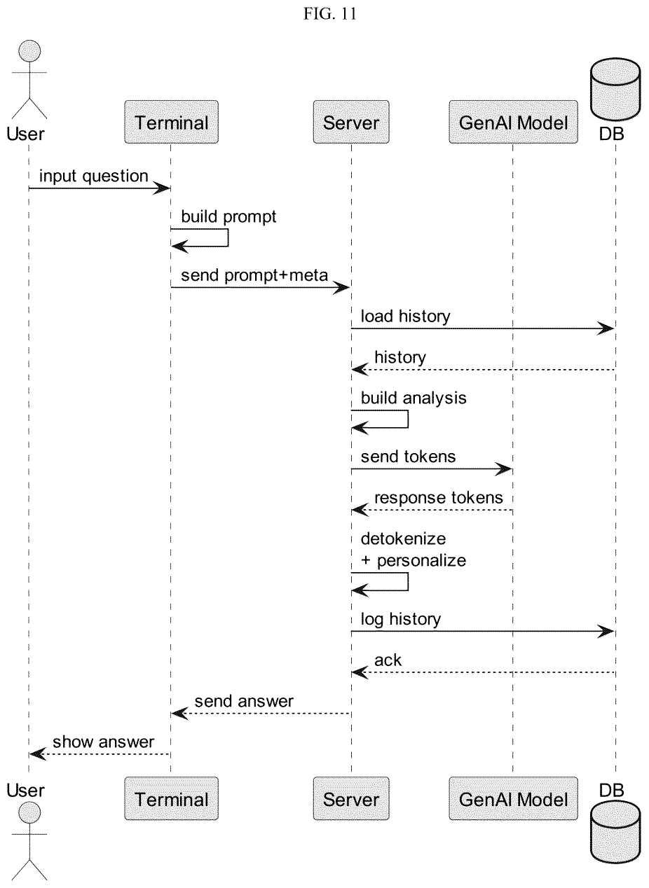

The following describes the processing flow using FIG. 11.

### Step 1:

The user operates the terminal to launch an animal-care application and to input inquiry information about an animal. The input is a sequence of user keystrokes or touch events representing a natural-language question, such as “My 4-year-old dog has been sleeping much more than usual and is eating a bit less. What should I do?”. The terminal converts these events into a text string, stores the string in working memory, and displays the string in an input field. The output of this step is the confirmed inquiry text stored as a text object in the terminal memory.

### Step 2:

The terminal generates a standardized prompt sentence based on the inquiry text and stored animal profile data. The input is the inquiry text and metadata such as species, age, and prior registration data retrieved from local storage. The terminal concatenates template phrases, the profile values, and the inquiry text, performs string normalization (for example, trimming whitespace and unifying character encoding), and produces a single structured prompt sentence suitable for a generative AI model. The output is a normalized prompt sentence, for example: “The user has a 4-year-old dog. The dog has been sleeping more than usual and has a slightly reduced appetite over the last week. As a generative AI model, analyze possible causes related to health and behavior, and provide clear, step-by-step recommendations, including when to consult a veterinarian.”

### Step 3:

The terminal attaches structured metadata to the prompt sentence to form communication information. The input is the prompt sentence and attribute values such as animal type, age, sex, and housing conditions. The terminal encodes these attributes into a structured internal object, associates the object with the prompt sentence, and assigns keys or field names to each attribute. The terminal then serializes this object into a byte sequence suitable for network transmission, for example, using a text-based data representation. The output is communication information that includes the prompt sentence and compact attribute fields.

### Step 4:

The terminal transmits the communication information to the server over a communication network. The input is the serialized communication information and connection parameters such as the server address and security credentials. The terminal establishes or reuses a secure session using its networking stack, encapsulates the communication information in protocol headers, and sends packets over a wireless or wired interface. The output is a stream of network packets containing the communication information that arrives at the server's network interface.

### Step 5:

The server receives the communication information and reconstructs the higher-level data structure. The input is the stream of incoming packets at the server's network interface. The server reassembles the packets, verifies integrity and authentication information, and extracts the original serialized communication information. The server then parses the serialized representation into an internal object in memory, recovering the prompt sentence and associated metadata fields. The output is an in-memory representation of the communication information, including the prompt sentence and structured metadata.

### Step 6:

The server builds analysis information by combining the prompt sentence with animal-related history information retrieved from a storage device. The input is the in-memory communication object and an animal identifier obtained from the metadata. The server issues database queries to a relational engine, retrieves records such as past weights, activity logs, feeding logs, and past inquiries, and stores them in data structures such as arrays or maps.

The server then constructs analysis information by embedding summary descriptors of this history, for example, weight trends or average activity duration, into additional context text or separate fields linked to the prompt sentence. The output is analysis information that includes the prompt sentence and structured history descriptors.

### Step 7:

The server tokenizes the prompt portion of the analysis information in preparation for input to the generative AI model. The input is the text content of the prompt sentence (which may already incorporate a brief summary of history) and the tokenizer vocabulary. The server segments the text into subword units according to tokenizer rules, maps each subword to a token identifier using a lookup table, and constructs an ordered list of token identifiers and positional indices. The output is a sequence of token identifiers and positions that represent the prompt sentence numerically.

### Step 8:

The server constructs a model input tensor and sends it to the generative AI model for inference. The input is the sequence of token identifiers and positions, and hyperparameters such as maximum output length and decoding temperature. The server maps token identifiers to embedding vectors by indexing into an embedding matrix stored in accelerator memory, adds positional encodings, and concatenates these vectors to form a two-dimensional tensor.

The server passes this tensor and the hyperparameters to the generative AI model function on the accelerator. The output is a configured model input state ready for forward computation.

### Step 9:

The server, through the generative AI model, performs transformer-based computation to generate response tokens. The input is the model input tensor and the trained model parameters (weight matrices and bias vectors). The server executes a series of matrix multiplications, attention computations, and non-linear activations layer by layer, calculates probability distributions over the vocabulary for each next-token position, and selects token identifiers based on a decoding algorithm such as greedy selection or beam search. The output is an ordered sequence of generated token identifiers representing the model's response.

### Step 10:

The server converts the generated token sequence back into a natural-language response text.

The input is the sequence of token identifiers and the tokenizer's reverse mapping from token identifiers to text fragments. The server maps each identifier to its corresponding subword string, concatenates the subwords in order, performs detokenization steps such as removing special markers and adjusting spaces, and forms a complete text string. The output is response information in the form of a natural-language answer that addresses the user's inquiry in general terms.

### Step 11:

The server enriches the response information using detailed health and behavior information from the database. The input is the response text, the history information retrieved earlier, and predefined rule tables describing thresholds and associated messages. The server computes summary statistics (for example, percentage weight change, average daily exercise time, or frequency of a particular symptom) and compares them to threshold values. The server then inserts additional sentences or clauses into the response text at designated locations, such as after a general recommendation, using text templates that reference the computed statistics. The output is personalized response information that is tailored to the specific animal's historical data.

### Step 12:

The server formats the personalized response information for transmission and logging. The input is the personalized text and any structured fields such as recommended exercise duration or feeding amount. The server encapsulates the text and structured fields into a response object, attaches a timestamp, a model version identifier, and a correlation ID, and serializes the object into a transferable representation. The server also writes an entry to a log or history table that records the prompt sentence, the personalized response, and key metrics such as processing time. The output is a serialized response payload ready for transmission and an updated history record stored in the database.

### Step 13:

The server transmits the serialized response payload to the terminal over the network. The input is the serialized payload and the existing communication session context. The server constructs protocol headers, assigns sequence numbers, and sends the payload through its network interface to the terminal's address. The server may also update internal metrics such as the number of successful responses for monitoring purposes. The output is a stream of packets containing the personalized response payload that arrives at the terminal.

### Step 14:

The terminal receives the response payload and reconstructs the personalized response information. The input is the stream of incoming packets and the terminal's communication session state. The terminal reassembles the packets, verifies integrity and session validity, and extracts the serialized payload. The terminal then parses the payload into a response object, recovering the personalized response text and any structured recommendation fields.

The output is an in-memory representation of the personalized response and associated metadata stored in the terminal memory.

### Step 15:

The terminal generates display data from the personalized response information and presents it to the user. The input is the response text, structured recommendation fields, and a set of display rules that define layout and highlighting behavior. The terminal creates user interface components such as text labels, bullet lists, or warning indicators, maps important terms (for example, “urgent” or “see a veterinarian”) to highlight styles, and arranges the components within a view hierarchy. The terminal then renders this view hierarchy to the display and may optionally generate haptic or audio cues if a warning flag is set. The output is visible and/or audible guidance presented to the user and stored display state that the user can scroll or revisit.

### Step 16:

The user reviews the displayed personalized response and may provide follow-up input to refine or extend the interaction. The input is the displayed guidance including explanations, thresholds, and recommended plans. The user interprets this information, decides whether to perform specific actions such as increasing exercise or contacting a professional, and, if needed, enters an additional question or status update through the terminal input interface.

The output is new inquiry information that can restart the processing sequence, allowing the system to build and utilize an extended history of interactions for future prompt sentences.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented pet care systems typically treat sensor data collection, rule-based analysis, and user communication as separate and loosely coupled functions. In many implementations, sensor data obtained from wearable devices or environmental sensors is processed by fixed-threshold algorithms, and static advice templates are sent to a user terminal. Such architectures present several technical problems.

First, the processing pipeline from sensor acquisition to advice presentation is often fragmented across different subsystems, resulting in increased latency, redundant data conversions, and inefficient use of processing resources. For example, raw sensor signals are preprocessed on a terminal, partially reprocessed on a server, and then reformatted multiple times before being rendered on a display device. This fragmentation can cause delays and inconsistencies in real-time monitoring scenarios where prompt feedback is important.

Second, existing systems typically do not leverage a generative AI model as an integrated component of the data-processing pipeline. Generative models, when used, are often invoked in an ad hoc manner with manually constructed prompts that do not systematically reflect the latest sensor-derived indices or history-based deviations. As a result, the computing device fails to exploit structured feature computation (such as health state indices and behavioral indices) to guide the generative AI model in a technically coherent way. This leads to suboptimal use of computational resources and can require repeated API calls or manual tuning, thereby reducing system responsiveness and determinism.

Third, most systems do not incorporate an automatic prompt-construction mechanism that dynamically converts preprocessed and feature-extracted sensor data into machine-readable and context-rich prompt sentences. Without such a mechanism, the processor must rely on static text patterns or developer-crafted prompts, which are not easily adaptable to varying sensor conditions, pet profiles, and historical patterns. This limits the scalability and maintainability of the software architecture and can degrade the accuracy and consistency of AI-generated evaluations and advice.

Fourth, conventional user interaction flows do not integrate emotion state analysis of the user into the core processing pipeline. User-facing responses from computational models are generally uniform, regardless of the emotional state or stress level of the user. This can result in ineffective communication, higher cognitive load, and repeated user queries, which in turn increase network and processing load in the overall system.

Fifth, there is a technical challenge in coordinating a wearable visual information presentation apparatus, server-side feature extraction, generative AI inference, and emotion analysis into a unified, low-latency system. Without a coordinated control mechanism on the server, the wearable device may not receive timely, context-adapted information, and the rendering pipeline on the wearable device may be underutilized or overburdened with non-prioritized data.

Accordingly, there is a need for a computer-implemented system in which a processor centrally manages acquisition of animal-related sensor information from a wearable visual information presentation apparatus, performs structured preprocessing and feature extraction on the sensor information, automatically constructs prompt sentences that encode health and behavior indices as inputs to a generative AI model, and generates optimized, emotion-aware responses that are transmitted back to the wearable device for real-time display. Such a system should technically improve the way computing resources are orchestrated, reduce redundant processing steps, lower interaction latency, and enhance the adaptability and robustness of AI-driven advisory output in response to dynamic sensor conditions and user emotional states.

The specific processing by the specific processing unit 290 of the data processing device 12 in Application Example 1 is realized by the following means.

The present invention provides a server comprising a processor, a storage apparatus, a communication apparatus, and access to a generative AI model, wherein the processor is configured to orchestrate acquisition of detection information from a wearable visual information presentation apparatus, perform preprocessing and feature extraction on the detection information and historical information stored in the storage apparatus to calculate a health state index and a behavioral state index, automatically generate structured prompt sentences including the indices and attribute information of an animal, provide the prompt sentences to the generative AI model to obtain an evaluated health state and at least one of advice information, training plan information, and nutrition management information, and further adjust at least one of the prompt sentences and the generative AI model responses based on emotion state analysis of an animal keeper before transmitting the adjusted information to the wearable visual information presentation apparatus for real-time display. This enables an integrated and technically improved computing pipeline in which sensor data collection, feature computation, prompt sentence generation for a generative AI model, emotion-aware response adaptation, and low-latency rendering on a wearable device are centrally controlled by the server, thereby reducing redundant processing, improving system responsiveness and resource utilization, and enhancing the consistency and effectiveness of

AI-generated guidance in real-time pet care support.

The term “processor” refers to a hardware-based information processing element, such as a central processing unit or a computation circuit, that executes instructions to perform data acquisition control, data preprocessing, feature extraction, prompt construction, model invocation, and response generation in the system.

The term “storage apparatus” refers to a hardware-based storage element, such as a memory device or a non-volatile storage device, that stores detection information, historical information, attribute information of an animal, and program instructions used by the processor.

The term “communication apparatus” refers to a hardware-based communication element, such as a network interface device or a wireless communication device, that transmits and receives information between the server, the wearable visual information presentation apparatus, and an external network.

The term “wearable visual information presentation apparatus” refers to a body-mountable information output apparatus, such as a head-mounted display or glasses-type display device, that includes a display unit for visually presenting information to an animal keeper and a detection apparatus for acquiring detection information.

The term “detection apparatus” refers to a sensing element or a combination of sensing elements, such as a sensor or an imaging element, that acquires detection information related to a physical state and a behavioral state of an animal.

The term “detection information” refers to digitally representable information acquired by the detection apparatus, including physical state information such as temperature-related information and behavioral state information such as movement-related information of an animal.

The term “historical information” refers to time-series information previously acquired and stored in the storage apparatus, including past detection information, past health state information, and past behavioral state information for an animal.

The term “attribute information of an animal” refers to predefined descriptive information about an animal, including at least one of species, age, body size, sex, and known health conditions, which is stored in the storage apparatus and used by the processor.

The term “health state index” refers to a computed indicator value or set of values derived by the processor from detection information and historical information, representing a quantified health condition of an animal.

The term “behavioral state index” refers to a computed indicator value or set of values derived by the processor from detection information and historical information, representing a quantified behavioral condition or activity level of an animal.

The term “deviation degree” refers to a computed value indicating a difference between a current or recent health state index or behavioral state index and a reference state derived from past health state information and past behavioral state information for an animal.

The term “generative AI model” refers to a machine-executable information processing model, such as a neural network model, that generates output information including natural-language text in response to input information including prompt sentences.

The term “prompt sentence” refers to a structured natural-language instruction or query generated or received by the processor and provided as input to the generative AI model, the prompt sentence including at least one of detection information, indices, attribute information, deviation degree information, and inquiry content.

The term “advice information” refers to natural-language information generated by the generative AI model that describes recommended care actions or operational guidance for an animal keeper with respect to an animal.

The term “training plan information” refers to natural-language information generated by the generative AI model that describes a set of training or activity procedures for modifying or improving behaviors of an animal over a time period.

The term “nutrition management information” refers to natural-language information generated by the generative AI model that describes recommended feeding amounts, schedules, or nutritional composition for maintaining or improving the health of an animal.

The term “emotion state analysis processing” refers to processing performed by the processor or by a cooperating computation element to estimate or classify an emotional state of an animal keeper based on input information such as voice information, text information, or interaction patterns.

The term “emotional state of the animal keeper” refers to an estimated psychological condition of the animal keeper, such as calm, anxious, stressed, or confused, determined by the emotion state analysis processing.

The term “response information” refers to natural-language text or structured content generated by the generative AI model in response to a prompt sentence, including at least one of evaluated health state, advice information, training plan information, and nutrition management information.

The term “display unit” refers to a visual output component of the wearable visual information presentation apparatus that presents characters, symbols, or graphical content to the animal keeper.

The term “time-series manner” refers to a presentation mode in which information is displayed in accordance with chronological order or update order so that an animal keeper can recognize changes over time.

In one embodiment, a system comprises a server, a terminal implemented as a wearable visual information presentation apparatus, and a communication network connecting the server and the terminal. A user acts as an animal keeper and wears the terminal to monitor an animal in real time.

The server includes a processor, a memory device, a non-volatile storage apparatus, and a communication interface. The terminal includes a head-mounted display, at least one detection apparatus such as a temperature sensor and a motion sensor, a local processor, a local memory, and a wireless communication module. The communication network may include a wireless local area network and a wide area network.

The terminal uses a hardware platform comparable to smart glasses, for example, a head-mounted display driven by a mobile processor running an operating system such as an Android-based platform or an embedded real-time operating system. The terminal uses a temperature sensor (for example, an infrared temperature sensor) and an inertial measurement unit (for example, a three-axis accelerometer and a three-axis gyroscope) as the detection apparatus. The terminal acquires analog or low-level digital signals from these sensors and converts the signals into digital detection information by using an integrated analog-to-digital conversion circuit and sensor driver modules provided by the operating system.

The terminal uses a display engine such as a graphics processing unit integrated on the mobile processor to render text and graphical overlays in a binocular or monocular display unit. The terminal executes an application that continuously collects detection information from the sensors, formats the detection information into structured internal data, and transmits the detection information to the server via the wireless communication module using a protocol such as Transmission Control Protocol and Hypertext Transfer Protocol over a secure channel.

The server uses a hardware platform based on a multi-core central processing unit and at least one graphics processing unit. The server executes a general-purpose operating system such as a Unix-like operating system. On top of the operating system, the server executes an application framework such as a web application framework and numerical computation libraries such as a matrix computation library. The server also executes a machine learning framework such as a tensor computation library for neural networks to host or access a generative AI model.

The server stores program code and model parameters of the generative AI model in the storage apparatus. The generative AI model in one embodiment is implemented as a transformer-based neural network having multiple encoder and decoder blocks. Each block includes a multi-head self-attention mechanism, a feed-forward network, and layer normalization. The model parameters include weight matrices, bias vectors, and layer normalization parameters, which are stored as floating-point tensors in the storage apparatus.

The server loads these parameters into the memory device at runtime and performs inference on the graphics processing unit.

The server uses a dedicated data structure to store detection information received from the terminal. For example, the server stores each detection record as an entry containing a timestamp, an identifier of the animal, a body temperature value, activity-related vector values derived from the motion sensor, and additional metadata such as sensor reliability flags. The server uses a time-series database schema or indexed tables in a relational database management system to store historical information for each animal.

The server uses numerical libraries to perform preprocessing on the detection information.

The server applies signal processing techniques such as moving average filtering and outlier removal based on statistical thresholds. For example, the server calculates a sliding average of body temperature over a fixed window and discards values that deviate from the window average beyond a defined standard deviation multiple. The server also calculates an activity level index by integrating acceleration magnitude over a time interval and normalizing the integrated value with respect to a baseline derived from historical information for the same animal.

The server calculates a health state index by combining the normalized body temperature, the rate of change of body temperature, and historical temperature statistics specific to the animal species and age. The server calculates a behavioral state index by combining the activity level index, time-of-day context, and deviations from typical daily activity profiles stored in the historical information. The server may represent each index as a multi-dimensional vector whose elements correspond to different sub-indicators, such as “thermal stress component,” “rest-activity ratio component,” and “short-term fluctuation component.”

The server constructs a prompt sentence for the generative AI model using a template engine.

The server maps numerical indices and attribute information of the animal into natural language segments. For example, the server generates a prompt sentence such as:

“You are a veterinary assistant AI. Species: dog. Age: 3 years. Current body temperature: 38.6° C. Average body temperature in the last 2 hours: 38.4° C. Temperature trend: slightly increasing. Current activity index (0-10): 6.2, which is above the usual level for this time of day. Evaluate whether the health status is normal or risky, and return a short explanation and a risk level (low, medium, high).”

The server passes this prompt sentence to the generative AI model via the machine learning framework. The server encodes the prompt sentence into token identifiers by using a tokenizer associated with the model. The server supplies the token sequence to the transformer-based neural network, and the network performs a series of matrix multiplications, attention score computations, and non-linear activations to generate a sequence of output token probabilities. The server decodes the output token probabilities into natural language text representing an evaluated health state and a risk level.

The server also constructs additional prompt sentences for advice generation. For example, the server generates a prompt sentence such as:

“You are a veterinary care advisor AI. The health evaluation for this dog is: ‘temperature normal, low health risk, slightly increased activity.’ The dog has been playing outdoors for about 30 minutes. Provide brief real-time care advice to the owner wearing smart glasses. Use 2-3 simple English sentences and focus on hydration and rest.”

The server invokes the same or another generative AI model with this prompt sentence to obtain advice information. The server can further generate prompt sentences for training plan information and nutrition management information, such as:

“You are a pet training expert AI. The dog's activity level has been low for the past week compared to its usual baseline. Suggest a 3-day light exercise plan suitable for a healthy 3-year-old dog. Use short, clear instructions in English.”

“You are a veterinary nutrition assistant AI. The dog weighs 12 kilograms and should maintain its current weight. Activity level is moderate. Suggest a daily feeding schedule, portion size guidelines, and snack limitations. Provide concise advice that a non-expert owner can follow.”

The server performs emotion state analysis on the user. In one embodiment, the terminal captures voice input from the user when the user speaks questions or commands. The terminal transmits audio data or transcribed text data to the server. The server uses an emotion classification model implemented as a neural network trained on features such as pitch variation, speech rate, pause duration, and word choice to estimate an emotional state such as calm, anxious, or stressed. The server stores the estimated emotional state in association with the current interaction session.

The server adjusts the prompt sentences and the generative AI model responses based on the emotional state. For example, when the user is estimated to be anxious, the server modifies the prompt sentence to instruct the generative AI model to use reassuring language and to provide more detailed explanations. The server may use a prompt sentence such as:

“You are a veterinary care advisor AI. The owner appears anxious. Provide a calm and reassuring explanation of the dog's current health status and clear step-by-step instructions.

Avoid technical jargon and focus on what the owner should do in the next 30 minutes.”

The server thus systematically encodes both sensor-derived indices and emotion-related context into prompt sentences, leading to consistent behavior of the generative AI model and reducing the need for manual prompt engineering.

The server transmits the generated health evaluation, advice information, training plan information, and nutrition management information back to the terminal via the communication interface. The server formats the information into a data packet in which fields correspond to distinct logical content segments, such as “summary,” “risk level,” and “immediate action steps.” The terminal receives the data packet, parses the content segments, and uses the local display engine to render the segments in distinct regions of the display. The terminal may present short, high-priority advice in a central region of the display and additional details in a peripheral region or in a secondary screen.

The user observes the information in real time and adjusts care actions accordingly, such as providing water, relocating the animal to a cooler place, or scheduling a visit to a veterinary facility. Because the server performs centralized processing of detection information, feature extraction, prompt construction, model inference, and emotion-aware adjustment, the system reduces redundant data transformations that would occur if each subsystem independently processed the data. The server uses vectorized operations on the graphics processing unit and batched model calls for multiple animals, thereby improving processing throughput and reducing latency compared to a design in which each interaction required a separate, manual prompt and isolated inference.

The server improves computer technology by introducing a non-conventional integration of time-series feature computation and automatic prompt construction for a generative AI model. In conventional systems, rule-based modules or static templates process sensor data and then humans write prompts or scripts for AI models. In contrast, the server automatically encodes computed indices, deviation degrees, and emotion states into a structured prompt sentence. This design reduces the number of required model invocations, minimizes unnecessary text processing by concentrating context in a single prompt, and allows the server to reuse intermediate feature representations across multiple advisory outputs. As a result, the system reduces computational load on the graphics processing unit and decreases communication overhead between application modules.

The server also improves the accuracy of health state evaluation by training the generative AI model on domain-specific training data that includes labeled examples of sensor patterns and corresponding veterinary assessments. During training, the server or an external training platform uses a loss function such as a cross-entropy loss that penalizes incorrect health risk classifications, and applies gradient-based optimization to update model parameters. The training process may include data augmentation techniques such as adding small perturbations to temperature and activity profiles and randomly masking context elements in prompt sentences to increase robustness. By aligning model outputs with structured indices in the prompt, the model learns to interpret numeric and categorical features more reliably than in a purely free-form conversational setting.

The server may employ additional algorithmic improvements to reduce errors. For example, the server can apply a post-processing step in which the generated text is checked against numeric consistency rules, such as verifying that a statement “temperature is normal” is compatible with species-specific normal temperature ranges stored in the storage apparatus.

When the generated text conflicts with numeric thresholds, the server can either request regeneration from the model using an amended prompt sentence specifying the inconsistency, or automatically correct the statement. This rule-based consistency layer further reduces erroneous outputs and improves reliability.

In another embodiment, the server hosts multiple generative AI models with different sizes or architectures, such as a smaller model for quick preliminary screening and a larger model for detailed explanation. The server dynamically selects which model to invoke based on current system load, network conditions, and the estimated urgency of the situation. For example, when the detection information indicates a rapidly increasing body temperature, the server may prioritize low-latency inference by using the smaller model and limiting the generated output to essential safety advice. This dynamic model selection reduces overall response time and prevents network congestion, thereby improving technical performance of the system.

The terminal may include additional detection apparatus, such as a camera for capturing images of the animal. The terminal can capture an image stream and transmit compressed image frames to the server. The server can run a convolutional neural network to extract visual features indicating posture, gait, or visible signs of distress. The server can then derive visual behavior indices and include them as elements in the health state index and behavioral state index. The inclusion of multi-modal features in the prompt sentence allows the generative AI model to consider richer context, improving the precision of its evaluations and recommendations.

The system is not limited to a particular species or sensor configuration. The server can store species-specific baseline ranges and automatically adjust preprocessing and index computation algorithms according to a species identifier. For example, the temperature normalization function and activity baseline profiles may differ between a dog and a cat. The server can maintain separate parameter sets for each species in the storage apparatus. The same generative AI model can then interpret species-specific indices by reading explicitly labeled information in the prompt sentence.

The server, terminal, and user cooperate to realize a system that is tightly coupled to physical sensors and display hardware, rather than a purely abstract information processing method.

The server directly controls how detection information is acquired from the terminal, how the graphics processing unit is used for model inference, and how the terminal's display engine is driven to present information in real time. By structuring the processing pipeline around indices, deviation degrees, and emotion-aware prompts, the server reduces computational redundancy, increases throughput, improves consistency of AI-generated guidance, and allows the user to manage animal health more effectively than with conventional fragmented systems.

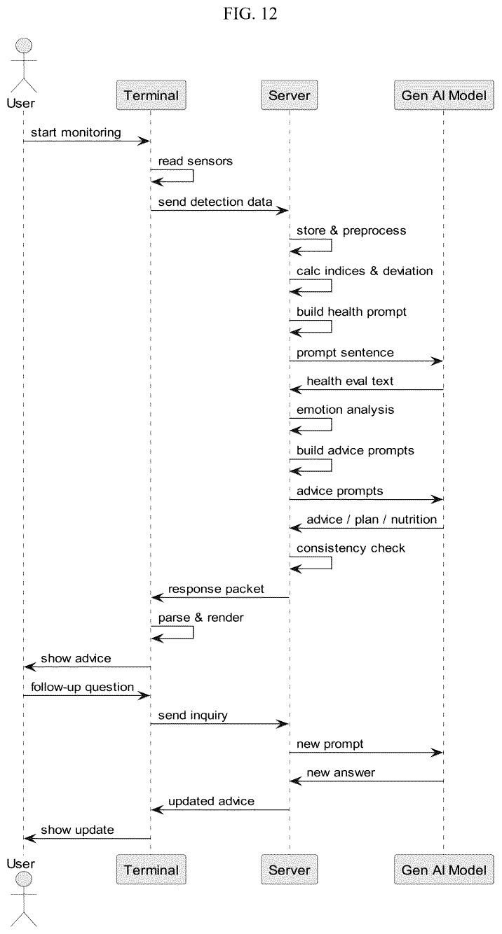

The following describes the processing flow using FIG. 12.

### Step 1:

The user wears the terminal and starts a monitoring application for an animal.

The input is a user operation such as power-on, application launch, or voice command.

The output is an active monitoring session state within the terminal.

The terminal changes its internal state from idle to monitoring mode, allocates memory buffers for sensor data, and initializes sensor drivers provided by an operating system framework.

### Step 2:

The terminal activates a detection apparatus and acquires raw sensor signals related to a physical state and a behavioral state of the animal.

The input is control parameters such as sampling rate, sensor type (temperature sensor, motion sensor), and sensor configuration data.

The output is a stream of raw sensor samples containing analog-to-digital converted values for temperature and motion.

The terminal uses an analog-to-digital conversion circuit to convert analog voltages from the temperature sensor into numeric values, and uses an inertial measurement unit interface to read acceleration and angular velocity values at fixed time intervals.

### Step 3:

The terminal converts the raw sensor samples into structured detection information.

The input is the raw sensor sample stream from the temperature sensor and the motion sensor.

The output is a data record including at least a timestamp, an animal identifier, a body temperature value, and motion feature values.

The terminal performs data processing such as unit conversion (for example, converting sensor counts to degrees Celsius), computation of acceleration magnitude from three-axis acceleration components, and time stamping by using a system clock. The terminal then stores these values into a structured data object.

### Step 4:

The terminal transmits the structured detection information to the server via a wireless communication module.

The input is the structured detection information produced in Step 3 and network configuration information such as server address and security credentials.

The output is a network message containing the detection information delivered to the server.

The terminal encodes the structured data into a transmission format, opens a communication channel using a communication protocol, and sends the message to the server's communication interface.

### Step 5:

The server receives the detection information and stores it as historical information in a storage apparatus.

The input is the network message arriving from the terminal, containing the detection information.

The output is a stored record in a time-series dataset associated with the animal.

The server parses the received message, validates the integrity of the fields, associates the detection information with an animal identifier and a session identifier, and writes the validated data into a database or similar storage structure.

### Step 6:

The server preprocesses the detection information and computes feature values for a health state index and a behavioral state index.

The input is the newly received detection information and relevant historical information for the same animal retrieved from the storage apparatus.

The output is a set of feature values including a normalized temperature value, a temperature trend value, an activity level index, and other intermediate indicators.

The server applies data processing such as filtering to remove outliers, moving average smoothing, differentiation to obtain rates of change, and normalization using species-specific baseline ranges. The server combines the processed values to form numerical vectors that represent the health state index and the behavioral state index.

### Step 7:

The server calculates a deviation degree from a reference state for the animal based on the indices and historical information.

The input is the health state index, the behavioral state index, and reference baseline profiles computed from long-term historical information in the storage apparatus.

The output is a deviation degree value or vector indicating differences between current indices and reference baseline values.

The server performs operations such as subtraction between current indices and baseline indices, and computes metrics such as Euclidean distance or standardized scores to quantify how far the current state deviates from normal patterns.

### Step 8:

The server constructs a first prompt sentence for the generative AI model to evaluate a health state of the animal.

The input is the health state index, the behavioral state index, the deviation degree, and attribute information of the animal such as species and age.

The output is a natural-language prompt sentence describing the current condition and requesting a health evaluation.

The server uses a template engine or string assembly logic to insert numeric values, categorical labels, and context information into predefined sentence templates, generating text such as:

“You are a veterinary assistant AI. Species: dog. Age: 3 years. Current body temperature: 38.6° C. Average body temperature in the last 2 hours: 38.4° C. Temperature trend: slightly increasing. Current activity index (0-10): 6.2, above the usual level for this time of day.

Evaluate whether the health status is normal or risky, and return a short explanation and a risk level (low, medium, high).”

### Step 9:

The server performs health-state evaluation by invoking the generative AI model with the first prompt sentence.

The input is the first prompt sentence constructed in Step 8.

The output is a model response containing an evaluated health state and a risk level expressed in natural language.

The server tokenizes the prompt sentence into token identifiers, feeds the token sequence to the generative AI model hosted on a processing unit such as a graphics processing unit, executes neural network inference to compute output token distributions, and decodes the output token distributions into text such as “The dog's body temperature is within the normal range for an adult dog. Health risk is low at this time.”

### Step 10:

The server performs emotion state analysis on the user to estimate an emotional state.

The input is user interaction data, such as audio recordings, transcribed text of user questions, and interaction timing logs received from the terminal.

The output is an emotional state label or score indicating, for example, calm, anxious, or stressed.

The server extracts acoustic features (such as pitch and speech rate) or linguistic features (such as use of urgent words) and applies an emotion classification algorithm implemented as a trained neural network or another classifier to compute probabilities for candidate emotional states, and then selects the most probable state as the emotional state of the user.

### Step 11:

The server constructs one or more second prompt sentences for generating advice information, training plan information, and nutrition management information, taking the emotional state into account.

The input is the evaluated health state and risk level from Step 9, the emotional state from Step 10, and the indices and deviation degree from previous steps.

The output is at least one natural-language prompt sentence configured for advice generation.

The server assembles text that integrates the evaluated health state, a description of the emotional state, and instructions to control the tone and level of detail, for example:

“You are a veterinary care advisor AI. The health evaluation for this dog is: ‘temperature normal, low health risk, slightly increased activity.’ The owner appears anxious. Provide a calm and reassuring explanation and clear step-by-step advice for the next 30 minutes. Avoid technical jargon.”

The server similarly creates prompt sentences for training and nutrition, requesting specific formats and constraints.

### Step 12:

The server generates advice information, training plan information, and nutrition management information by invoking the generative AI model with the second prompt sentences.

The input is the set of second prompt sentences constructed in Step 11.

The output is a set of natural-language texts containing concrete advice, training steps, and nutrition recommendations.

The server forwards each prompt sentence to the generative AI model, runs inference, and decodes the outputs into text segments, such as “Offer fresh water and allow the dog to rest in a cool place for 10-15 minutes” or “Provide two meals per day of approximately 120 grams each, and limit treats to once per day.”

### Step 13:

The server applies post-processing and consistency checks to the generated texts and prepares a response packet for the terminal.

The input is the generated advice information, training plan information, nutrition management information, and reference rules such as species-specific normal ranges stored in the storage apparatus.

The output is a structured response packet containing validated text segments and associated metadata.

The server analyzes the generated texts for internal consistency with numeric thresholds, corrects or regenerates parts that conflict with stored rules, attaches priority tags (for example, “urgent,” “informational”), and organizes the segments into fields that can be rendered separately by the terminal.

### Step 14:

The server transmits the response packet to the terminal through the communication interface.

The input is the structured response packet prepared in Step 13 and connection parameters to the terminal.

The output is a delivered network message containing the response packet at the terminal side.

The server selects an appropriate communication protocol, serializes the packet into a transmittable format, and sends the packet over the network connection to the terminal's network endpoint.

### Step 15:

The terminal receives the response packet and parses the contained information for display.

The input is the network message containing the response packet from the server.

The output is a set of display elements such as text strings and priority indicators ready for rendering on the wearable display unit.

The terminal decodes the message, verifies integrity, separates the advice text, training text, and nutrition text into individual variables, and determines display layout positions and styles based on priority tags and user preferences.

### Step 16:

The terminal displays the received information on the wearable visual information presentation apparatus for the user.

The input is the parsed display elements from Step 15 and display configuration data (font size, position, color).

The output is a visual presentation of real-time advice, health evaluation summaries, and optional training and nutrition guidance in the user's field of view.

The terminal instructs a graphics processing unit to draw text overlays on the head-mounted display, updates the content as new packets arrive, and may highlight urgent items with distinct colors or icons to attract the user's attention.

### Step 17:

The user observes the displayed information and optionally issues follow-up questions or commands.

The input is the visual information presented on the terminal and the user's perception and decision.

The output is user actions such as care actions applied to the animal or new queries input via voice or gesture.

The user may ask, for example, “Is it safe to continue playing for another 20 minutes?” The terminal captures the voice, converts it to text, and sends the text as new inquiry content to the server, starting a new cycle of processing based on updated detection information and user questions.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional animal-care assistance systems mainly provide static rule-based guidance or simple threshold alerts based on sensor readings. Such systems typically collect activity data or feeding logs and trigger notifications when predefined limits are exceeded. However, these systems suffer from several technical limitations in terms of computer technology itself.

First, existing architectures do not perform integrated, structured preprocessing of heterogeneous biological information and behavioral information (for example, multi-dimensional sensor signals and image streams). Raw data are often stored or analyzed in an ad hoc manner without systematic signal processing, normalization, or segmentation. This leads to noisy inputs for downstream algorithms, lowers the accuracy and robustness of behavior estimation, and increases processing load on the processor because later components must compensate for poor data quality.

Second, conventional systems generally do not utilize generative AI models in a structured, prompt-driven way for behavior analysis and advice generation. Even when machine learning is used, models are trained for narrow classification tasks and are not coupled with dynamically configured prompt sentences that encode behavior evaluation information, animal attributes, and target activity levels. As a result, the processor cannot flexibly generate context-aware, personalized management instruction information, and the computational pipeline cannot be easily adapted to different animals, conditions, and time scales without manual reprogramming.

Third, existing systems typically ignore the emotional state of the user when generating and presenting guidance. The user interface layer often displays fixed-format messages irrespective of the user's current emotional condition. From a computer-technology perspective, this means that the system fails to use available multimodal inputs (interactive text and image information) to compute an internal emotional-state representation and adjust prompt sentences and generated outputs. Consequently, the processor does not optimize the interaction flow or presentation style, which reduces the effectiveness of the generated content and can lead to repeated unnecessary requests, additional server load, and inefficient use of communication and processing resources.

Fourth, prior architectures rarely incorporate user feedback on generated management instruction information as structured data to update internal computation conditions.

Evaluation information and additional input information from the user, if collected at all, are not systematically used to adjust prompt configurations or recalibrate calculation conditions for behavioral evaluation information. This limits the system's ability to continuously improve its internal models and processing pipeline, and prevents the processor from optimizing its resource allocation and inference pathways based on real-world usage.

Fifth, many systems lack a coordinated mechanism in which the processor aggregates historical behavioral evaluation information and management instruction information over predetermined periods, and then automatically generates summary reports via generative AI models. Without such temporal aggregation and automated reporting, servers must execute repeated ad hoc queries and formatting routines, leading to redundant computations, increased memory usage, and fragmented user interactions.

Accordingly, there is a need for an improved computer-implemented system in which a processor: (i) acquires and preprocesses heterogeneous biological and behavioral information into structured time-series and image information sets; (ii) inputs these sets into a generative AI model to obtain consistent behavioral evaluation information; (iii) constructs and updates prompt sentences that integrate evaluation results, animal attributes, and target activity levels; (iv) computes management instruction information and summary reports through the generative AI model; (v) estimates a user's emotional state to adapt prompt sentences and presentation content; and (vi) incorporates user feedback to update internal prompt configurations and calculation conditions. Such a system improves the functioning of the computer itself by enabling more accurate, efficient, and adaptive processing of multimodal animal-related data and user interaction data.

The specific processing by the specific processing unit 290 of the data processing device 12 in Example 2 is realized by the following means.

The present invention provides a server comprising a processor configured to acquire biological information and behavioral information from at least one detection device and at least one imaging device associated with an animal, associate the biological information and behavioral information with time information, and store the associated information in a storage device; perform, by executing signal-processing and statistical-processing instructions, abnormal value removal, interpolation processing, normalization processing, and interval segmentation processing on the biological information and the behavioral information to generate at least one time-series information set and at least one image information set suitable for analysis; input the time-series information set and the image information set to a generative AI model implemented on a machine-learning framework and executed by the processor, and estimate behavioral evaluation information including at least an activity index, a rest-state index, a feeding-behavior index, and an abnormal-behavior index of the animal; generate management instruction information including at least one of an exercise plan, a training plan, a nutrition-management plan, and a medical-consultation recommendation by configuring a prompt sentence based on the behavioral evaluation information, attribute information of the animal, and target activity-level information, and by inputting the prompt sentence to the generative AI model to obtain the management instruction information expressed in natural language; perform emotion-analysis processing by using the processor to estimate an emotional state of a user from interactive input information and image information, and adjust at least one of an expression format of the prompt sentence and a presentation content of the management instruction information in accordance with the emotional state; transmit the management instruction information and the adjusted presentation content to an information terminal via a communication network to cause the information terminal to present notification information; and store evaluation information or additional input information received from the information terminal with respect to the management instruction information, and update at least one of a configuration of the prompt sentence and calculation conditions of the behavioral evaluation information based on the evaluation information or the additional input information. This enables the server to improve computer functionality by transforming unstructured multimodal animal-related data and user interaction data into structured, model-ready representations, performing more accurate and adaptive behavior evaluation via the generative AI model, dynamically tailoring prompt sentences and generated outputs to the user's emotional state, and incrementally refining internal computation parameters based on user feedback, thereby reducing processing redundancy, improving resource utilization, and enhancing the technical quality and effectiveness of automated animal-care guidance.

The term “processor” refers to a hardware-based computation unit, such as a central processing unit or a graphics processing unit, that executes machine-readable instructions to perform data acquisition, data processing, model inference, and communication control in the system.

The term “detection device” refers to an electronic sensing apparatus, such as an accelerometer, a motion sensor, or a physiological sensor, that is configured to detect biological information or behavioral information related to an animal and to output corresponding digital signals.

The term “imaging device” refers to an electronic image-capturing apparatus, such as a video camera or a still-image camera, that is configured to capture visual information related to an animal and to output corresponding image data.

The term “biological information” refers to measurement data representing a physical or physiological state of an animal, including but not limited to motion-related signals, posture-related signals, and vital-sign-related signals acquired by a detection device.

The term “behavioral information” refers to measurement data representing an action, activity, or behavior pattern of an animal, including but not limited to movement sequences, feeding actions, resting actions, and other observable behaviors derived from detection device data and imaging device data.

The term “time information” refers to temporal data, such as timestamps or time indices, that indicate when specific biological information or behavioral information was acquired, and that enable chronological association and ordering of such information.

The term “storage device” refers to a hardware-based data retention component, such as a semiconductor memory or a magnetic storage unit, that is configured to store biological information, behavioral information, time information, intermediate processing results, and generated content under control of the processor.

The term “abnormal value removal” refers to a data-processing operation in which outlier values or implausible measurements are detected according to a predetermined criterion and removed or replaced to improve the reliability of the biological information and the behavioral information.

The term “interpolation processing” refers to a data-processing operation in which missing data points or gaps in time-series biological information or behavioral information are estimated and filled based on surrounding data according to a predetermined interpolation method.

The term “normalization processing” refers to a data-processing operation in which numerical ranges, scales, or distributions of biological information or behavioral information are transformed into standardized forms, such as rescaling or mean-variance normalization, to facilitate subsequent analysis.

The term “interval segmentation processing” refers to a data-processing operation in which continuous biological information or behavioral information is divided into discrete time intervals or segments according to a predetermined temporal window or event-based rule.

The term “time-series information set” refers to a structured collection of biological information or behavioral information arranged in temporal order, segmented into intervals, and formatted as numerical sequences suitable for input to a machine-learning model.

The term “image information set” refers to a structured collection of image data or image-derived features obtained from an imaging device, processed into a standardized spatial and temporal format suitable for input to a machine-learning model.

The term “machine-learning framework” refers to a software-based environment or library that provides functions for defining, training, and executing machine-learning models, including generative AI models, on the processor.

The term “generative AI model” refers to a machine-trained computational model that is configured to generate output data, such as natural-language text or inferred behavior patterns, based on input data and learned parameters, using a machine-learning framework.

The term “behavioral evaluation information” refers to analysis results obtained by the generative AI model, including numerical indices, categorical labels, and indicators that quantify or characterize an animal's activity, rest, feeding behavior, and abnormal behavior.

The term “activity index” refers to a numerical indicator included in the behavioral evaluation information that represents a level or intensity of physical activity of an animal over a predetermined period.

The term “rest-state index” refers to a numerical indicator included in the behavioral evaluation information that represents a level, duration, or quality of rest or sleep of an animal over a predetermined period.

The term “feeding-behavior index” refers to a numerical indicator or categorical descriptor included in the behavioral evaluation information that represents feeding-related behavior, such as appetite level, feeding duration, or irregular feeding events of an animal.

The term “abnormal-behavior index” refers to a numerical indicator or categorical descriptor included in the behavioral evaluation information that represents a likelihood, degree, or type of abnormal behavior exhibited by an animal, such as possible illness or allergy-related behavior.

The term “attribute information” refers to descriptive data of an animal individual, including but not limited to species, breed, age, body mass, known medical conditions, and environmental conditions registered in the system.

The term “target activity-level information” refers to reference data that define a desired or recommended range or pattern of physical activity or behavior for an animal, based on attribute information or external guidance.

The term “management instruction information” refers to generated guidance content for a user, including at least one of an exercise plan, a training plan, a nutrition-management plan, and a medical-consultation recommendation derived from behavioral evaluation information and other data.

The term “exercise plan” refers to a portion of the management instruction information that specifies recommended physical activities for an animal, including types, durations, frequencies, and intensities of such activities.

The term “training plan” refers to a portion of the management instruction information that specifies recommended behavioral training activities or routines for an animal, including tasks, repetition schedules, and reinforcement strategies.

The term “nutrition-management plan” refers to a portion of the management instruction information that specifies recommended feeding-related actions for an animal, including types of food, amounts, feeding schedules, and dietary restrictions.

The term “medical-consultation recommendation” refers to a portion of the management instruction information that indicates whether and when a user should seek professional medical or veterinary consultation for an animal, based on behavioral evaluation information.

The term “prompt sentence” refers to a structured natural-language or machine-readable text string that encodes input conditions, context information, and requested tasks, and that is provided as input to the generative AI model to cause generation of output content.

The term “natural language” refers to a human language expression, such as textual sentences, that can be read and understood by human users without requiring specialized encoding or programming syntax.

The term “interactive input information” refers to user-provided data obtained through an interface, including but not limited to text messages, voice-transcribed content, selection operations, and other inputs that are used for dialog and control within the system.

The term “image information” refers to visual data obtained from an imaging device or derived from such data, including pixel values, feature maps, or compressed image representations used for analysis.

The term “emotion-analysis processing” refers to a computation procedure executed by the processor in which interactive input information and image information are analyzed to estimate an emotional state of a user, using predetermined rules or learned models.

The term “emotional state” refers to an internal representation within the system of a user's affective condition, such as calm, anxious, confused, or stressed, estimated through emotion-analysis processing.

The term “expression format” refers to a style or manner of phrasing, structuring, or formatting generated text, including tone, politeness level, level of detail, and ordering of information in the management instruction information or response content.

The term “presentation content” refers to a subset or arrangement of generated information selected for display to a user on an information terminal, including which elements are emphasized, summarized, or suppressed.

The term “information terminal” refers to a user-operated electronic device, such as a mobile device or a computing device, that communicates with the server via a communication network and presents notification information or other content to the user.

The term “communication network” refers to a wired or wireless data communication infrastructure, such as a local network or a wide-area network, that enables data transmission between the server, detection devices, imaging devices, and information terminals.

The term “notification information” refers to presentation data transmitted from the server to an information terminal to inform a user of newly generated management instruction information, reports, or other system events.

The term “evaluation information” refers to user-provided feedback data regarding generated management instruction information, including indications of usefulness, correctness, compliance, or user satisfaction.

The term “additional input information” refers to supplementary user-provided data related to the animal or the management instruction information, including comments, corrections, or updated animal conditions.

The term “calculation conditions” refers to parameters, thresholds, weights, or configuration settings that control how the processor computes behavioral evaluation information or other intermediate analysis results.

The term “history of the behavioral evaluation information” refers to a temporally ordered collection of behavioral evaluation information records accumulated over multiple time periods for an animal.

The term “history of the management instruction information” refers to a temporally ordered collection of management instruction information records generated and provided to a user over multiple time periods.

The term “predetermined period” refers to a time interval specified by the system or user, such as a day, a week, or a month, used for aggregating behavioral evaluation information and management instruction information.

The term “summary information” refers to aggregated or condensed data that represent key trends, statistics, or notable events derived from the history of the behavioral evaluation information and the history of the management instruction information.

The term “report sentence” refers to a natural-language text generated by the generative AI model that describes, explains, or summarizes the summary information for presentation to a user.

The term “response content” refers to a natural-language answer or explanation generated by the generative AI model in reply to question content contained in interactive input information.

The term “tone” refers to a stylistic attribute of the response content or management instruction information, such as formality level, friendliness, urgency, or reassurance, which can be adjusted according to the emotional state.

The term “level of detail” refers to a degree of granularity or comprehensiveness in the response content or management instruction information, including how much explanation, background, or step-by-step guidance is included.

The term “presentation order” refers to a sequence or arrangement in which elements of the response content or management instruction information are presented to the user, such as ordering of main recommendations, warnings, and explanations.

In one embodiment, a server, a terminal, and a user cooperate to implement an animal-management system that executes the claimed functions using concrete hardware and software components.

### A. Hardware and Software Configuration

The server includes at least one central processing unit, at least one graphics processing unit, a main memory, a non-volatile storage device, and a network interface. The server executes an operating system such as a general-purpose server operating system, and executes application programs implemented, for example, in a high-level programming language. The server further executes a machine-learning framework such as a general-purpose deep learning library in order to implement a generative AI model as described below.

The server is connected via a communication network to at least one detection device, at least one imaging device, and at least one terminal. The detection device is implemented as a wearable sensor module attached to an animal, and may include a three-axis accelerometer, a gyroscope, a temperature sensor, and a wireless communication module. The imaging device is implemented as a network camera installed in a living environment of the animal and outputs a video stream via a standard streaming protocol. The terminal is implemented as a mobile communication device or a general-purpose computing device having a display, an input interface, a wireless communication module, and a storage. The terminal executes an application that communicates with the server over a secure protocol.

The server uses a relational or non-relational database management system, such as a general-purpose relational database or a time-series database, for storing biological information, behavioral information, time information, analysis results, and user feedback. The server also uses an object storage system for storing image data and video-derived data. For numerical computation, the server uses a numerical computation library and a signal-processing library.

For image processing, the server uses an image-processing library. For orchestration of periodic tasks, the server uses a scheduler.

### B. Data Structures and Acquisition

The server acquires biological information and behavioral information from the detection device and the imaging device. The server stores the biological information in a time-series data structure. For example, the server stores samples of accelerometer readings in records having fields including a timestamp, acceleration values along three axes, a device identifier, and a pet identifier. The server stores the behavioral information derived from the imaging device as image frames or video segments along with metadata including capture time, frame index, device identifier, and pet identifier.

The server associates the biological information and the behavioral information with time information by aligning sensor timestamps with frame timestamps. The server stores the resulting data in a storage device in a manner that permits efficient retrieval for a specified time interval and a specified pet identifier. The data structure is designed such that the server can retrieve sequences of sensor samples and corresponding image frames with a single indexed query, thereby reducing access latency and I/O operations.

### C. Preprocessing and feature construction

The server performs abnormal value removal, interpolation processing, normalization processing, and interval segmentation processing on the biological information and the behavioral information. The server uses a signal-processing library to apply a digital filter to the accelerometer signals to remove high-frequency noise and sensor glitches. The server uses statistical criteria, such as thresholding based on standard deviation or interquartile range, to identify and remove outlier values that exceed plausible physical limits. The server applies interpolation processing, such as linear interpolation or spline interpolation, to fill short gaps caused by temporary communication loss. The server then performs normalization processing, for example, by subtracting the mean and dividing by the standard deviation for each signal dimension over a defined window.

The server segments the continuous biological information into fixed-length intervals, such as 60-second windows with a specified overlap. Each segment is represented as a two-dimensional array having a sample dimension and a feature dimension. The server segments the behavioral information from the imaging device into sequences of frames corresponding to these intervals and resizes each frame to a fixed spatial resolution. The server converts each frame to a standardized color space and constructs tensors representing a sequence of frames.

By performing this structured preprocessing and segmentation, the server improves the quality and uniformity of input data provided to the downstream generative AI model. This reduces computational burden in the model, stabilizes training and inference, and improves the accuracy of behavior estimation compared to using unfiltered, irregularly sampled, and unaligned data.

### D. Generative AI Model Architecture and Training

The server implements a generative AI model using a machine-learning framework. In one embodiment, the generative AI model includes multiple sub-networks: a time-series encoder, an image encoder, and a language-generation decoder.

The server implements the time-series encoder as a neural network such as a bidirectional recurrent neural network, a temporal convolutional network, or a transformer-based encoder.

The time-series encoder receives a time-series information set as input and outputs an embedding vector that represents activity dynamics over the interval. The server implements the image encoder as a convolutional neural network or a vision transformer that receives an image information set and outputs an embedding vector representing visual behavior patterns, such as feeding posture, scratching behavior, or rest posture.

The server concatenates or otherwise combines the time-series embedding and the image embedding into a joint representation. The server passes this joint representation to a behavioral evaluation head constructed as a feedforward neural network that outputs behavioral evaluation information including an activity index, a rest-state index, a feeding-behavior index, and an abnormal-behavior index. These indices can be represented as continuous values, probability distributions, or multi-label outputs.

The server trains the behavioral evaluation part of the generative AI model using supervised learning. The server uses labeled training data that include annotated activity levels, rest episodes, feeding events, and abnormal behaviors from multiple animals. During training, the server minimizes an objective function such as a weighted sum of mean squared error for continuous indices and cross-entropy loss for categorical outputs. The server updates network weights using a gradient-based optimization algorithm such as stochastic gradient descent or Adam. The server may also apply data augmentation methods, for example, temporal jittering of sensor segments and random cropping or horizontal flipping of image frames, to increase robustness and reduce overfitting.

For natural-language generation, the server implements a language-generation decoder as a sequence-to-sequence model, for example, a transformer-based decoder. The server trains the decoder using pairs of structured inputs (behavioral evaluation information, attribute information, target activity-level information, and other context) and target natural-language descriptions or advice texts. The server uses an autoregressive training objective, minimizing a negative log-likelihood loss over target tokens. The server may fine-tune a pre-trained language model with domain-specific data to improve fluency and relevance.

By jointly designing the architecture to process heterogeneous inputs and generate structured evaluation outputs and natural-language advice, the server creates a computational system that performs a specialized technical task that would be difficult to implement using rule-based logic alone. The neural-network structures exploit statistical regularities in high-dimensional time-series and image data to yield more accurate and robust behavioral evaluation information than conventional threshold-based or manually engineered feature approaches.

### E. Prompt Sentence Construction and Language Generation

The server constructs a prompt sentence that encodes behavioral evaluation information, attribute information of the animal, and target activity-level information. The server uses deterministic rules to compose prompt sentences in a structured template form to reduce variability and improve reliability of generative responses. For example, the server may construct a prompt as follows:

“You are a virtual veterinary assistant. Analyze the following pet behavior and generate personalized advice for the owner.

Pet profile: 3-year-old, 10-kg dog, no known chronic diseases, moderate activity is recommended.

Last 7 days activity index (0-100): [45, 40, 38, 35, 30, 32, 28]. Target index: 60.

Feeding behavior: the pet eats all meals and shows no vomiting or diarrhea. No signs of food allergy were detected by the behavior model.

Task: Explain whether the exercise level is sufficient and propose a realistic 1-week exercise plan the owner can follow, with daily walking times and play activities.”

The server then inputs this prompt sentence to the generative AI model. When the model is integrated end-to-end, the same generative AI framework uses the joint representation from the behavioral encoders as a conditioning vector for the language decoder. When the model is separated, the server passes the prompt sentence to a standalone language model interface. In both cases, the server obtains a natural-language response that constitutes management instruction information, such as an exercise plan, a training plan, a nutrition-management plan, or a medical-consultation recommendation.

Because the prompt sentence explicitly encodes numerical indices, trends, and target levels in a machine-readable but human-interpretable format, the generative AI model can learn to map specific patterns in behavioral evaluation information to structured recommendations.

This structured prompt design reduces ambiguity, narrows the search space of generated outputs, and thereby improves generation speed and prediction stability compared to unstructured free-text prompts.

### F. Emotion-Analysis Processing and Adaptation of Outputs

The server performs emotion-analysis processing to estimate an emotional state of the user from interactive input information and image information. For example, the server can receive text messages from the user via the terminal, and apply a text-classification neural network that produces probabilities for emotional categories such as calm, anxious, confused, or frustrated. The server may also receive facial images from the terminal and apply a separate convolutional neural network trained to detect facial expressions indicative of emotional states.

The server combines probabilities from text-based and image-based classifiers using a weighting rule or a small fusion network to determine an overall emotional state representation. The server then adjusts at least one of an expression format of the prompt sentence and a presentation content of the management instruction information based on this representation. For example, if the user is estimated to be anxious, the server modifies the prompt sentence by including explicit instructions to generate more reassuring, step-by-step explanations. Similarly, the server can suppress technical jargon and emphasize clear, concise directions in the generated advice. If the user is estimated to be calm and experienced, the server may request a more concise and technical summary.

In one embodiment, the server modifies the prompt sentence by adding a clause such as:

“The owner currently appears anxious. Please answer in a reassuring tone, avoid technical jargon, and provide step-by-step instructions.”

or, alternatively:

“The owner is an experienced user and currently appears calm. Please provide a concise, technical summary with minimal repetition.”

By adjusting prompt sentences and downstream outputs according to a computed emotional state, the server improves user comprehension and reduces repeated follow-up queries. This, in turn, reduces redundant model invocations and avoids unnecessary data transmissions, thereby lowering communication load and processing overhead, and improving the technical performance of the system as a whole.

### G. Feedback Incorporation and Dynamic Updating of Computation Conditions

The server receives evaluation information and additional input information from the terminal. The user can indicate whether the advice was useful, whether it was followed, and what outcomes were observed. The server stores this information in association with the underlying behavioral evaluation information and prompt configuration.

The server uses the accumulated feedback data to update calculation conditions, such as thresholds used in aggregating activity indices, or weightings used to balance different behavioral indices when forming summary metrics. The server may also refine the configuration of prompt sentences. For instance, if feedback indicates that a particular phrasing or level of detail leads to user confusion, the server can adjust the templates used to create prompt sentences for similar situations.

The server can implement a learning module that adjusts internal hyperparameters using an optimization routine based on prediction errors with respect to user-reported outcomes. This allows the system to automatically calibrate itself to different classes of animals, environments, and user behaviors, thereby improving prediction accuracy and communication efficiency over time.

### H. Summary Report Generation

The server aggregates a history of behavioral evaluation information and management instruction information for predetermined periods such as a week or a month. The server computes summary statistics, including average activity index, variance of rest-state index, frequency of abnormal-behavior indices exceeding a threshold, and distribution of nutrition-management changes.

The server constructs a prompt sentence that describes these summaries, for example:

“Create a monthly health and activity report for the following pet. Pet: 4-year-old indoor cat, 4 kg, no chronic illnesses. Over the last 30 days, daily activity index ranged from 20 to 60, with an average of 35, which is below the recommended level of 50. Feeding logs show that the pet left 20-30% of food uneaten on 10 days. Two possible food allergy events were detected, but the owner reported that changing to hypoallergenic food improved the symptoms.

Generate a clear, owner-friendly report summarizing the pet's health, activity patterns, feeding behavior, and provide practical recommendations for the next month, including when a veterinary check is advisable.”

The server inputs this prompt sentence to the generative AI model and obtains a report sentence that summarizes the period. The server provides the report sentence to the terminal.

The structured aggregation and automated text generation reduce repeated ad hoc query and formatting operations. This leads to improved computational efficiency, reduced memory consumption, and smoother user interaction.

### I. Operation of the Terminal and User Interaction

The terminal communicates with the server to receive management instruction information and report sentences. The terminal presents these outputs in a user interface that can include text sections, graphical plots of indices over time, and alert icons. The terminal also captures interactive input information from the user, including questions and feedback, and transmits these to the server for further processing.

The user operates the terminal to register pets, confirm device pairing, view advice, submit feedback, and optionally provide additional context such as observations or veterinarian diagnoses. The cooperative actions of the server, terminal, and user enable the system to continuously refine its internal models and computation conditions.

### J. Technical Effect and Improvement of Computer Technology

The described system does not merely automate human interpretation of animal behavior.

Instead, the system introduces a specific data architecture, preprocessing pipeline, and multi-modal neural-network model that together improve the functioning of the computer system itself.

By converting heterogeneous, noisy sensor and image data into normalized and segmented time-series and image information sets, the server reduces downstream computational complexity and improves the stability and accuracy of inference. The use of specialized neural architectures for time-series and image data, combined with structured prompt sentences linking behavioral evaluation information to generative language output, allows the system to perform a complex mapping that is not achievable by conventional rule-based or threshold-based systems with comparable efficiency or accuracy.

Furthermore, by incorporating emotion-analysis processing into the control of prompt sentences and presentation content, the server reduces unnecessary repeated requests and network traffic, thereby improving communication efficiency and overall system throughput.

The feedback-based adaptation of calculation conditions and prompt configurations enables self-calibration of the computational pipeline, which lowers prediction error over time and optimizes resource usage.

Alternative embodiments may vary the specific neural architectures, learning algorithms, or data-aggregation strategies, while maintaining the core concepts of structured preprocessing, multi-modal generative AI modeling, prompt-based control of natural-language outputs, emotion-adaptive interaction, and feedback-driven recalibration. For example, the server may replace a recurrent time-series encoder with a purely attention-based encoder, or may employ different loss functions to handle imbalanced behavior classes. The server may also employ alternative image encoders or incorporate additional sensor modalities such as audio. All such variations are intended to be encompassed within the scope supported by the claims.

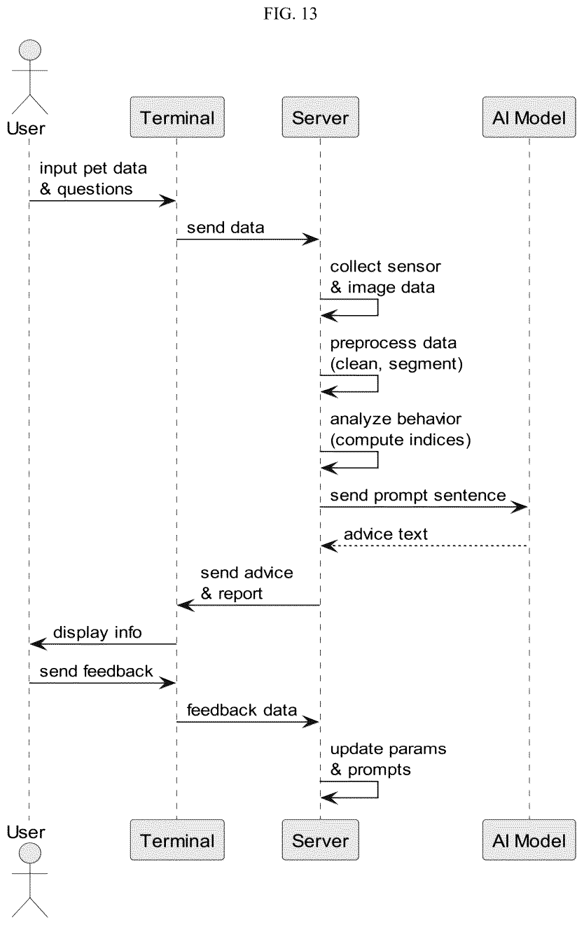

The following describes the processing flow using FIG. 13.

### Step 1:

Server acquires raw biological information and behavioral information.

Server uses a communication interface to receive sensor data from the detection device and image data from the imaging device.

Input: raw accelerometer readings, gyroscope readings, temperature values, and video frames or image files, each with device identifiers and timestamps.

Server associates each incoming sample with a pet identifier and time information, and writes the raw data into a time-series database for sensor values and an object storage system for image files.

Output: stored raw sensor records and stored raw image files, each indexed by pet identifier, device identifier, and timestamp.

### Step 2:

Server retrieves and aligns time-series and image data for a specified analysis period.

Server receives a request (internally scheduled or triggered) specifying a pet identifier and a time interval.

Input: pet identifier, start time, end time.

Server queries the time-series database to load all sensor records in the interval and queries metadata in the storage system to load corresponding image file paths.

Server aligns sensor samples and image frames by interpolating or snapping timestamps to a common temporal grid so that each time step can reference both sensor values and optional image data.

Output: aligned sequences of raw sensor samples and associated image frame references for the specified period.

### Step 3:

Server performs abnormal value removal on the aligned sensor data.

Input: aligned raw sensor sequences, including accelerometer and optional gyroscope or temperature values with timestamps.

Server computes basic statistics (such as mean and standard deviation) or uses predefined physical limits to identify outlier values; for example, server flags acceleration magnitudes that exceed a maximum threshold as abnormal.

Server removes or replaces flagged samples using neighboring values or interpolation.

Output: cleaned sensor sequences in which outlier samples have been removed or corrected, maintaining time alignment.

### Step 4:

Server performs interpolation processing and normalization processing on the cleaned sensor data.

Input: cleaned sensor sequences with possible missing samples due to removal or communication gaps.

Server applies interpolation algorithms, such as linear interpolation between nearest valid samples, to estimate missing sensor values at required timestamps.

Server then normalizes each sensor channel by subtracting a window-based mean and dividing by a window-based standard deviation, or by applying min-max scaling to a fixed range.

Output: continuous, normalized sensor sequences with consistent sampling intervals and scale, suitable for machine-learning input.

### Step 5:

Server segments the normalized time-series into fixed-length intervals and constructs time-series information sets.

Input: normalized continuous sensor sequences over the analysis period.

Server divides the sequences into contiguous windows of a predetermined length (for example, 60 seconds), optionally overlapping by a fixed stride (for example, 30 seconds).

Server packs each window into a two-dimensional array whose rows correspond to time steps and whose columns correspond to sensor channels; server attaches metadata such as pet identifier, start time, and window index.

Output: a collection of time-series information sets, each representing a fixed-length segment with standardized size and metadata.

### Step 6:

Server preprocesses image data to form image information sets.

Input: image frame file paths associated with the analysis period and their timestamps.

Server reads each image file from object storage, resizes the image to a fixed resolution, converts the color space to a standard format, and optionally applies background subtraction or motion detection to emphasize the animal's region.

Server groups frames into sequences aligned with the time-series windows (for example, frames whose timestamps fall within a given 60-second window) and converts each frame into a numerical tensor.

Output: a collection of image information sets, each comprising a sequence of processed image tensors aligned with a corresponding time-series information set.

### Step 7:

Server encodes time-series information and image information using neural-network encoders.

Input: time-series information sets and image information sets for each window.

Server loads a trained time-series encoder network, such as a temporal convolutional or transformer-based model, and forwards each time-series information set through the encoder to obtain an activity representation vector.

Server loads a trained image encoder network, such as a convolutional network or vision transformer, and forwards each image information set through the encoder to obtain a visual behavior representation vector.

Server concatenates or otherwise fuses the activity representation vector and visual behavior representation vector to form a joint embedding for each window.

Output: joint embedding vectors representing combined sensor and image behavior for each window.

### Step 8:

Server computes behavioral evaluation information from the joint embeddings.

Input: joint embedding vectors for all windows in the analysis period, plus optional contextual features such as time of day.

Server applies a behavioral evaluation head implemented as a fully connected neural network that maps each embedding to predicted indices: an activity index, a rest-state index, a feeding-behavior index, and an abnormal-behavior index.

Server aggregates window-level indices into period-level metrics by computing statistics such as mean, maximum, and duration above or below thresholds; server also flags abnormal patterns (for example, persistent low activity or recurring post-meal discomfort).

Output: behavioral evaluation information for the period, including aggregated indices and flags per pet identifier.

### Step 9:

Server constructs a prompt sentence incorporating behavioral evaluation information and animal attribute information.

Input: behavioral evaluation information for a specified period, attribute information (species, age, body mass, known conditions), and target activity-level information for the animal.

Server fills a prompt template with the indices, trends, and targets, creating a structured natural-language context; for example, server inserts the last seven days of activity indices and the target index.

Server adds an explicit task description specifying what the generative AI model shall produce, such as advice on exercise, training, nutrition, or medical consultation.

Output: a prompt sentence in natural-language text form that encodes evaluation results, context, and requested output type.

### Step 10:

Server generates management instruction information by sending the prompt sentence to a generative AI model.

Input: constructed prompt sentence and, when applicable, additional conditioning vectors derived from the behavioral encoders.

Server invokes a generative AI model implemented in a machine-learning framework, passing the prompt sentence as input to a language-generation decoder; server configures parameters such as maximum output length and sampling temperature.

Server receives generated text from the decoder, parses it as management instruction information that may contain an exercise plan, training plan, nutrition-management plan, and medical-consultation recommendation.

Output: natural-language management instruction information tailored to the specific animal and its recent behavior.

### Step 11:

Server estimates a user emotional state from interactive input and optionally image information.

Input: recent user interactive input information, such as text questions or comments submitted from the terminal, and optional user image information, such as facial images captured by the terminal camera.

Server feeds the text input into a text-based emotion classifier neural network to obtain probabilities for multiple emotional categories; server optionally feeds facial images into a facial-expression classifier network to obtain an independent estimate.

Server combines these estimates using a fusion rule, such as a weighted average or a small neural network, to derive a final emotional state representation, which can be a probability vector or a discrete label.

Output: an emotional state estimate associated with the current user interaction session.

### Step 12:

Server adapts the prompt sentence and presentation content based on the emotional state.

Input: original prompt sentence, generated management instruction information, and emotional state estimate.

Server modifies the prompt sentence or constructs an additional control phrase that instructs the generative AI model about desired tone, level of detail, or style; for example, when the user is anxious, server adds a clause to request a reassuring, step-by-step explanation.

Server may also select or reorder segments of the management instruction information so that critical safety-related recommendations are highlighted or presented first, according to the emotional state and system rules.

Output: an adjusted prompt sentence and adjusted presentation content that are emotion-aware and optimized for user understanding.

### Step 13:

Server transmits adjusted management instruction information to the terminal.

Input: adjusted presentation content of management instruction information, pet identifier, and user identifier.

Server packages the information into a response message and sends it over a secure communication channel to the terminal; server may also generate a push notification payload containing a summary of the content and an identifier for retrieval.

Server records transmission metadata, such as timestamp and delivery status, for later auditing and performance analysis.

Output: transmitted message containing management instruction information, which is received by the terminal for display to the user.

### Step 14:

Terminal presents notification information and detailed guidance to the user.

Input: received notification payload and management instruction information from the server.

Terminal displays a concise notification to the user, indicating that new advice or a report is available; when the user opens the application, terminal requests and receives the full text if not already cached.

Terminal renders the management instruction information in a user interface, possibly including separate sections for exercise, training, nutrition, and medical advice, and graphical representations of indices over time.

Output: visual and optionally audio presentation of personalized guidance on the terminal screen, enabling the user to understand the system's recommendations.

### Step 15:

User reviews the guidance and optionally provides feedback or additional information.

Input: displayed management instruction information and graphical summaries on the terminal.

User reads the advice, compares it with observed animal behavior, and may interact with the interface to rate usefulness, indicate whether the plan will be followed, or enter comments about actual outcomes.

User may also enter additional context, such as recent veterinarian diagnoses or environmental changes, through text fields or selection options.

Output: user-generated feedback data and additional input information captured by the terminal.

### Step 16:

Terminal sends user feedback and additional input information to the server.

Input: feedback ratings, comments, compliance indicators, and additional context information entered by the user.

Terminal encapsulates these data into a structured message that includes identifiers for the related advice, pet, and time period, and sends the message to the server over a secure channel.

Terminal confirms successful transmission to the user or retries if a communication error occurs.

Output: transmitted feedback and additional input information stored in server-accessible form.

### Step 17:

Server updates internal calculation conditions and prompt configurations based on feedback.

Input: user evaluation information and additional input information linked to past management instruction information and behavioral evaluation information.

Server analyzes feedback patterns, for example, by computing correlations between reported outcomes and specific index thresholds or prompt templates; server may adjust parameters such as exercise-index cutoffs, weighting of abnormal-behavior indices, or default level of detail in responses.

Server stores updated calculation conditions and revised prompt templates in configuration storage, so that subsequent behavioral evaluation and prompt construction use refined settings that are better aligned with real-world results.

Output: updated internal configuration data that influence future processing, enabling improved prediction accuracy and more effective communication.

### Step 18:

Server aggregates historical behavioral evaluation information and generates periodic summary reports.

Input: histories of behavioral evaluation information and management instruction information over a predetermined period for a given pet.

Server computes aggregated statistics, trends, and key events (such as recurring low activity, frequent abnormal-behavior flags, or significant changes in feeding patterns); server then constructs a summary-oriented prompt sentence that describes these findings and instructs the generative AI model to create a report.

Server passes the summary prompt sentence to the generative AI model and receives a report sentence describing the period's health and behavior status with recommendations for the near future.

Output: natural-language summary report tailored to the animal and the selected period, ready for delivery to the terminal and presentation to the user.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional animal-care support systems that incorporate artificial intelligence generally treat a user's question as a simple text query and return template-based or statically generated answers. Such systems typically do not construct a structured computational context that combines (i) multi-modal, time-series sensor data representing an animal's behavior and health, (ii) historical records, and (iii) the user's current natural-language question, into a unified machine-interpretable representation. As a result, the underlying computing components perform fragmented processing, in which natural language processing, sensor analytics, and dialogue generation are executed in isolation, leading to sub-optimal use of computing resources and inconsistent output quality.

Moreover, in many existing systems, a generative AI model is invoked directly with the raw user question as input, without systematic generation of a task-specific prompt sentence that encodes the outcome of prior numerical analysis. In such architectures, the generative AI model internally re-infers context that could have been pre-computed, leading to redundant computation, increased latency, and unstable response behavior. This hinders predictable control over the AI model and complicates scaling and optimization of server-side processing pipelines.

Further, conventional systems rarely integrate computational emotion analysis into the core control loop of the generative AI model. Even when emotion or sentiment analysis is present, it is commonly used only as a superficial label attached to the output, rather than as a first-class signal that algorithmically adjusts prompt generation parameters, output formatting, and response timing. Consequently, the server fails to systematically adapt interaction strategies to the emotional state of the user, resulting in inefficient use of bandwidth and compute (e.g., sending overly verbose or overly terse responses) and reduced effectiveness of subsequent AI inferences that could otherwise leverage logged emotion-context correlations.

Additionally, prior systems generally log raw interactions in an ad-hoc manner, without recording, in a machine-readable form, the explicit association among (a) the generated prompt sentences given to the generative AI model, (b) the corresponding emotion information, and (c) the resulting responses. This deficiency prevents the server from algorithmically updating prompt generation rules or input conditions for the generative AI model based on accumulated interaction records. As a result, there is no systematic feedback loop that improves the computational behavior of the server over time, such as reducing average response latency, stabilizing answer style, or improving the relevance of generated content.

Therefore, there is a need for a computer-implemented system that (i) constructs and maintains a unified context representation combining time-series sensor analytics, historical records, and natural-language questions, (ii) generates structured prompt sentences for a generative AI model based explicitly on this context, (iii) computes and applies emotion information as a control signal to adjust output content and format, and (iv) persistently stores associations among prompts, emotion information, and responses so that prompt generation conditions and model input conditions can be updated. By addressing these issues as a computer-technology problem, the invention aims to improve the technical operation of server-side AI pipelines, including efficiency of resource utilization, determinism and controllability of generative AI behavior, and adaptability of the system based on logged interaction data.

The specific processing by the specific processing unit 290 of the data processing device 12 in Application Example 2 is realized by the following means.

The present invention provides a server comprising a processor configured to (i) analyze natural-language input information acquired from an animal caretaker together with attribute information related to an animal and time-series behavioral information related to the animal, and generate, based on the analysis, a structured prompt sentence to be supplied to a generative AI model; (ii) acquire observation information including biological information and behavioral information of the animal from a detection unit, record the observation information as time-series data, perform preprocessing on the time-series data including at least noise reduction, normalization, and temporal segmentation, and generate an analysis result indicating at least an activity amount, a behavioral tendency, and an abnormality degree; (iii) construct, based on the analysis result, history information related to the animal, and the natural-language input information, the prompt sentence to be input to the generative AI model, and convert response information acquired from the generative AI model into output information including at least advice information, training plan information, and nutrition management information for presentation to the animal caretaker; (iv) analyze voice information or character information of a user to calculate emotion information indicating an emotional state of the user, and adjust at least a content of expression and a presentation manner of the output information based on the emotion information; and (v) store, in a storage device, the output information and the emotion information in association with each other as record information, and update at least a generation condition for the prompt sentence or an input condition for the generative AI model based on the stored record information. This enables the server to implement a unified and technically improved AI processing pipeline in which sensor analytics, prompt sentence generation, emotion-aware response control, and feedback-based updating of model input conditions are integrated, thereby reducing redundant computation, improving latency and stability of generative outputs, and adaptively optimizing the behavior of the generative AI model over time based on accumulated interaction data.

The term “system” refers to an arrangement of hardware and software components, including at least one processing unit, memory, and communication interfaces, that cooperate to execute the functions described in the claims.

The term “processor” refers to a hardware data-processing unit, such as a central processing unit or an execution core, that executes machine-readable instructions to perform logical operations, arithmetic operations, data movement, and control operations.

The term “animal caretaker” refers to a human user who manages or supervises the health, behavior, or living environment of an animal, and who provides input to the system through a user interface.

The term “natural-language input information” refers to information expressed in a human language, such as text or speech content, that is acquired from the animal caretaker and processed by the system.

The term “attribute information related to an animal” refers to structured data describing inherent or static characteristics of an animal, including but not limited to species, age, weight, sex, medical history, and identification information.

The term “time-series behavioral information related to the animal” refers to data describing the animal's behavior over time, represented as sequential records each associated with at least one timestamp, such as movement patterns, activity levels, or posture changes.

The term “prompt sentence” refers to a machine-generated or machine-formatted natural-language instruction or description that is provided as input to a generative AI model to condition or guide the model's output.

The term “generative artificial intelligence model” refers to a computational model implemented using machine-learning or statistical techniques that, responsive to an input including at least a prompt sentence, generates new output data such as natural-language text.

The term “interactive processing” refers to processing in which the system and the animal caretaker exchange information in a turn-based or real-time manner, including question-and-answer dialogs or multi-turn conversations.

The term “detection unit” refers to a hardware unit including one or more sensors, imaging devices, or measurement devices that acquire observation information related to the animal's biological state or behavior.

The term “biological information of the animal” refers to data describing physiological or biometric aspects of the animal, such as heart rate, body temperature, respiratory rate, or other measurable bodily parameters.

The term “behavioral information of the animal” refers to data indicating actions or movements of the animal, including but not limited to walking, running, resting, sleeping, eating, or vocalizing.

The term “observation information” refers to a collection of one or more items of biological information and/or behavioral information obtained from the detection unit.

The term “time-series data” refers to data that consists of a plurality of values each associated with a time index or timestamp, allowing analysis of changes or patterns over time.

The term “noise reduction” refers to processing applied to time-series data to suppress or remove components that are considered artifacts, measurement errors, or random fluctuations, thereby improving the signal quality.

The term “normalization” refers to processing of numerical data that adjusts the scale or distribution of the data, such as by rescaling values to a predefined range or by standardizing values to have a desired statistical property.

The term “temporal segmentation” refers to processing that divides time-series data into multiple segments or windows based on time intervals or event boundaries for separate analysis.

The term “analysis result” refers to data produced by computational processing of observation information or time-series data, and that indicates one or more derived measures such as activity amount, behavioral tendency, or abnormality degree.

The term “activity amount” refers to a quantitative index representing a level of physical activity of the animal over a given time period, such as total movement duration, step count, or energy expenditure.

The term “behavioral tendency” refers to a characterization of typical or predominant behavior patterns of the animal inferred from time-series data, such as frequency of specific activities or distribution of active and resting periods.

The term “abnormality degree” refers to an index indicating a magnitude or likelihood of deviation of the animal's behavior or biological state from a predefined normal pattern or threshold.

The term “history information related to the animal” refers to stored information representing past states, events, or measurements associated with the animal, including past observation information, prior analysis results, and previously generated recommendations.

The term “response information” refers to data output from the generative AI model in response to input including at least a prompt sentence, such as generated natural-language text describing recommendations or explanations.

The term “output information” refers to information derived from the response information that is formatted or processed for presentation to the animal caretaker, and that may include advice information, training plan information, or nutrition management information.

The term “advice information” refers to information that recommends one or more actions or precautions for the animal caretaker to take regarding care, monitoring, or management of the animal.

The term “training plan information” refers to information that specifies one or more training activities or behavioral exercises for the animal, including at least scheduled actions, durations, or conditions.

The term “nutrition management information” refers to information that specifies or recommends feeding practices, dietary compositions, or nutritional adjustments intended to support or improve the animal's health.

The term “voice information of a user” refers to audio data representing spoken utterances of the user, acquired through a microphone or similar device.

The term “character information of a user” refers to text data representing the user's input, including typed characters, transcribed speech text, or other textual expressions.

The term “emotion information” refers to data indicating an estimated emotional state of the user, such as anxiety, calmness, stress, or satisfaction, computed from voice information. character information, or other user signals.

The term “content of expression of the output information” refers to the wording, tone, level of detail, structure, and style of the output information as perceived by the user.

The term “presentation manner of the output information” refers to how the output information is presented to the user, including modality, layout, ordering, emphasis, and timing of display or delivery.

The term “record information” refers to stored data that associates at least the output information with corresponding emotion information, and optionally includes the prompt sentence, the user input, and the analysis result.

The term “generation condition for the prompt sentence” refers to one or more rules, parameters, templates, or control values used by the processor to construct a prompt sentence to be provided to the generative AI model.

The term “input condition for the generative AI model” refers to one or more parameters, configuration values, or constraints that control how the system provides input to the generative AI model, including formatting, context selection, or inference settings.

The term “context information” refers to a combined set of data used to characterize a situation at a time of generating a prompt sentence or response, including at least a user question, time-series data, and history information.

The term “question sentence” refers to a natural-language expression provided by the animal caretaker that requests information, advice, or clarification from the system.

The term “answer information” refers to output information that is specifically structured as a response to a question sentence, and that incorporates expression adjustments based on emotion information.

The term “activity index” refers to a numerical or categorical indicator derived from the analysis result that summarizes the animal's activity amount over a given period.

The term “health state index” refers to a numerical or categorical indicator derived from the analysis result or history information that summarizes an estimated health condition of the animal.

The term “presentation timing” refers to a point in time or schedule at which the system provides output information to the user, including immediate display, deferred notification, or periodic delivery.

In one embodiment, a server executes a program that integrates sensor analytics, natural language processing, generative AI model control, and emotion-aware response adjustment in a unified processing pipeline. The server comprises at least one processor, a main memory, a non-volatile storage device, and a communication interface connected to a network. The server runs an operating system such as a general-purpose server operating system and executes application software implemented, for example, in a high-level programming language. The server stores a trained generative AI model, one or more neural network models for time-series sensor analysis, and one or more models or rule sets for emotion recognition.

The terminal comprises a mobile computing device such as a smartphone or tablet that includes at least one processor, a memory, a display, a microphone, and one or more sensors such as an accelerometer and a gyroscope. The terminal executes an application that acquires user input, collects sensor data related to an animal, and communicates with the server via a network using a protocol such as HTTPS. The terminal may further include a camera or an external wearable sensor interface so that motion data or biological data of the animal can be acquired.

The user operates the terminal to input natural-language questions or status reports regarding an animal. The user may type text through a keyboard interface or speak into the microphone, in which case the terminal applies on-device or remote speech-to-text processing to obtain character data. The user may also attach labels, such as “new food” or “visited park,” that are stored as structured metadata together with time stamps.

The terminal transmits to the server a data structure that includes at least the natural-language input, attribute information related to the animal, and recent time-series behavioral data. The attribute information may include species, breed category, age range, approximate weight range, and indications of known conditions or allergies. The time-series data may include motion vectors from the accelerometer and gyroscope, heart-rate samples from a wearable sensor, or other biological measurements. The terminal may compress or batch these data to reduce network overhead.

The server stores the received data into a persistent storage system, such as a relational data store, by mapping each interaction to a session record. The server writes a unified context record containing fields for the raw natural-language text, a normalized animal profile, references to time-series data blocks, and an interaction identifier. By employing a structured schema, the server enables subsequent modules to access the same context object instead of duplicating parsing or retrieval operations.

The server executes a text preprocessing module that converts the natural-language input into a normalized representation. The server applies tokenization, lowercasing, and punctuation normalization, and may apply lemmatization and stopword filtering. The server extracts key tokens and phrases indicating symptoms or behaviors, such as “less active,” “not eating,” or “barking at night,” using pattern matching and statistical keyword extraction.

The server executes a time-series analysis module that processes the behavioral information and biological information acquired from the detection unit. The server represents the time-series data as arrays of tuples (timestamp, feature vector). The feature vector may include three-axis accelerometer values, three-axis gyroscope values, and additional channels such as heart rate. The server performs noise reduction using, for example, a low-pass filter or a moving average filter applied per channel. The server performs normalization by scaling each channel to zero mean and unit variance over a configurable window, or by scaling values into a predetermined range.

The server divides the normalized time-series into fixed-length windows, such as 30-second or 60-second segments, for temporal segmentation. For each segment, the server computes descriptive features including mean and variance of each axis, energy, zero-crossing rate, and spectral features obtained by applying a discrete Fourier transform. The server aggregates these features into a fixed-length feature vector for each window.

The server feeds the window-level feature vectors into an activity classification neural network. In one example, the server uses a convolutional neural network (CNN) or a recurrent neural network (RNN) such as a long short-term memory (LSTM) network, implemented using a machine-learning framework. The network includes an input layer matching the feature vector dimension, one or more hidden layers with non-linear activation functions, and an output layer producing probabilities for activity classes such as walking, running, resting, sleeping, and atypical movement. The server obtains a sequence of class probabilities for each window and integrates them over the observation period to compute an activity amount index and a behavioral tendency index. For example, the server calculates total estimated active time, longest continuous rest interval, and frequency of high-intensity activity.

The server computes an abnormality degree by comparing the current activity indices and biological indices to a baseline derived from past data. The server stores historical summaries in the data store and computes moving averages and variances for each index. The server calculates deviations, for example as z-scores, and defines an abnormality degree as a function of the magnitude and duration of deviations beyond thresholds. This algorithmic comparison reduces false alarms and provides a quantifiable measure of change.

The server executes an emotion recognition module that processes user voice information or character information. When voice information is available, the server extracts acoustic features such as pitch contour, energy, speaking rate, and spectral features. When only character information is available, the server analyzes word choice, punctuation, and sentence length. The server applies a classifier such as a neural network with an embedding layer followed by recurrent or transformer layers, or a support vector machine using hand-crafted features. The classifier outputs a probability distribution over emotion categories such as calm, anxious, frustrated, or satisfied. The server converts this distribution into emotion information that includes both category labels and confidence values.

The server creates a unified context object that references: the parsed natural-language input; the attribute information; the activity amount index, behavioral tendency index, and abnormality degree; relevant history information such as previous episodes and prior recommendations; and the emotion information. The context object is stored in memory and is used as the basis for prompt generation.

The server generates a prompt sentence by applying prompt templates that encode system-level instructions to the generative AI model. The server fills template slots with textual descriptions derived from the context object. For example, the server generates a prompt sentence such as:

“You are a virtual assistant that supports an animal caretaker. Analyze the following pet data and the owner's question. Pet: animal type, approximate age, approximate weight, and known conditions. Recent activity summary: total active time, longest rest period, and overall abnormality degree. Owner question: ‘My dog has been less active today. Is there a problem?’ Provide concise, concrete advice for the caretaker, including when to consult a veterinarian.”

The server may generate other prompt sentences such as:

“Using the following 7-day activity summary, determine if this animal shows signs of chronic under-exercise or over-exercise and generate proactive advice for the caretaker.”

or

“Pet has recently reduced appetite and decreased activity. Explain possible non-urgent home checks and clearly indicate warning signs that require immediate veterinary attention.”

By explicitly encoding analyzed indices and history into the prompt sentence, the server offloads numerical interpretation from the generative AI model and reduces the need for the model to infer context from raw text, which improves computational efficiency and stabilizes the model behavior.

The server provides the generated prompt sentence, together with optional structured context, to the generative AI model. In one embodiment, the server uses a transformer-based language model that has been pretrained on large text corpora and optionally fine-tuned on domain-specific data. The model comprises multiple transformer blocks including self-attention layers, feed-forward layers, and normalization layers. The server tokenizes the prompt sentence into subword tokens, maps them to numerical embeddings, and feeds the token sequence through the transformer layers to obtain a probability distribution over the next token at each step. The server generates output tokens by iteratively sampling or selecting tokens according to configured decoding strategies.

The server adjusts model parameters such as temperature, top-k, and maximum token length based on interaction type. For example, the server may use a lower temperature and shorter maximum length when the abnormality degree or emotion information indicates a potential urgent situation, thereby producing more focused and less verbose responses. Conversely, for routine guidance with low abnormality degree and neutral emotion, the server may allow more expansive explanations.

The server post-processes the generated token sequence to obtain response information in natural language. The server formats the response into structured sections, such as a short summary, a list of concrete actions, and optional additional explanations. The server consults the emotion information and applies rule-based modifications to adjust tone and style. For example, when the emotion category is anxious with high confidence, the server adds reassuring phrases and simplifies technical terms. When the emotion category is calm, the server may include more detailed rationale.

The server converts the final response information into output information that is intended for display or audio playback on the terminal. The output information may include advice information describing specific actions, training plan information describing scheduled exercises, and nutrition management information describing feeding adjustments. The server encodes references to the underlying indices so that downstream components can trace which computational findings led to each recommendation.

The server stores, in association, the generated prompt sentence, the context object, the emotion information, the output information, and any subsequent user feedback such as ratings or follow-up questions. By logging this record information in a structured manner, the server can analyze patterns across interactions. The server periodically executes an optimization component that adjusts generation conditions for the prompt sentence, such as template choices, inclusion or exclusion of certain indices, and decoding parameter presets.

The optimization component may also tune input conditions for the generative AI model, including context window length and formatting strategies, based on measured outcomes such as user satisfaction indicators and error rates.

The server thereby improves computer technology in several ways. First, by separating numerical analysis of time-series data from textual generation while explicitly encoding numerical summaries into prompt sentences, the server reduces redundant inference within the generative AI model and lowers average processing time per request. Second, by using dedicated neural networks for activity recognition and emotion recognition, and feeding their outputs as structured features into the prompt construction process, the server increases the precision and stability of generated responses relative to systems that supply only raw user questions. Third, by maintaining a feedback loop where prompt generation parameters and model input parameters are updated based on accumulated record information, the server continuously improves resource utilization and response quality without requiring full retraining of underlying models.

The terminal receives the output information and renders it in a user interface. The terminal may highlight urgent content using visual indicators and may schedule local notifications based on presentation timing information included in the output. The terminal may store a local subset of record information so that the user can review past recommendations even without network connectivity. This local caching reduces network traffic and allows the server to skip redundant transmission of unchanged content.

In another embodiment, the server operates with an alternative neural network architecture for time-series analysis, such as a temporal convolutional network or a hybrid architecture that combines convolutional layers for local pattern extraction with recurrent layers for long-range dependencies. The server may also apply data augmentation techniques during model training, such as time-warping, noise injection, or random cropping of windows, to improve robustness against sensor noise. The server trains the time-series models using supervised learning with a loss function such as cross-entropy for activity classification, and trains the emotion recognition models using annotated datasets of text or speech with emotion labels.

In a further embodiment, the server deploys multiple generative AI models specialized for different domains, such as behavior, nutrition, or general wellness. The server selects among these models or composes multiple outputs based on analysis result categories and user preferences. The server may also support fallback rules: when abnormality degree exceeds a threshold or when sensor data are incomplete, the server automatically constrains the generative AI output by appending explicit instructions into the prompt sentence to recommend professional consultation rather than attempting detailed diagnosis.

The user interacts with the system by following recommendations and optionally providing feedback. The user may mark a recommendation as helpful or not helpful, or confirm that a particular problem was resolved. The server incorporates this feedback into its record information and can recalibrate thresholds used to compute abnormality degree or adjust which historical features are most predictive, thereby gradually improving the technical behavior of the analysis pipeline.

Because the server explicitly defines data structures for context objects, record information, and indices, and because the server employs specialized neural network models and algorithmic comparison of indices against baselines to generate prompt sentences, the system does not merely automate human reasoning but implements a computer-specific architecture that optimizes model usage, reduces latency, and improves reproducibility of AI-generated content. The described embodiments thus provide concrete technical improvements in the operation of a computer system that manages complex multimodal animal-care data and emotion-aware dialogue generation.

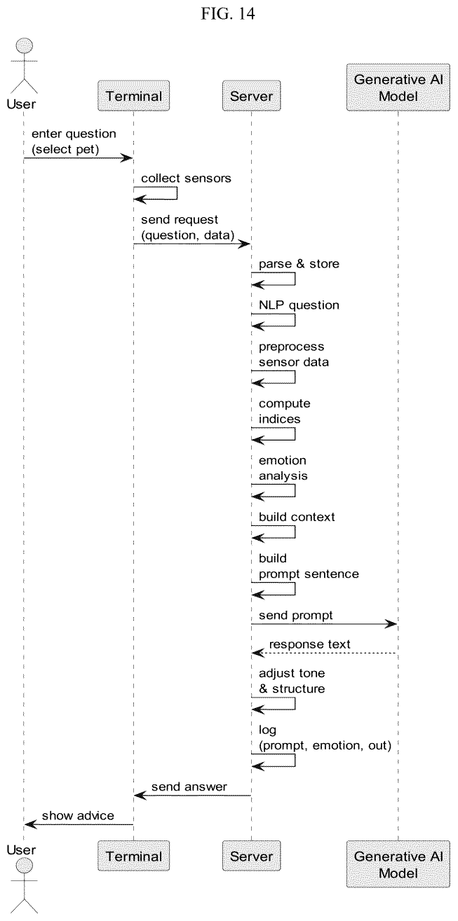

The following describes the processing flow using FIG. 14.

### Step 1:

User operates the terminal to input a natural-language question and animal context.

User types a question such as “My dog has been less active today. Is there a problem?” or speaks into the microphone, and may select the animal profile (species, approximate age, weight range) and optional tags such as “new food” or “visited park.”

Input: User's raw text or speech signal and selected animal profile.

Output: Terminal-internal structured data including a normalized text question, animal attribute fields, optional tags, and a timestamp.

Terminal converts speech to text using a speech-to-text engine, normalizes character encoding, and stores the result as a UTF-8 string in a data structure that also contains identifiers for the animal and the user.

### Step 2:

Terminal collects and packages recent sensor data for the animal.

Terminal acquires motion data from built-in sensors such as an accelerometer and a gyroscope, or from connected wearable devices, for a defined recent period (for example, the last several hours).

Input: Raw sensor readings (timestamp, sensor axis values, optional heart-rate samples) acquired by the terminal or received from a wearable.

Output: A time-series buffer containing an ordered list of records, each record including a timestamp and a feature vector of sensor channels.

Terminal aggregates sensor readings into a buffer, ensures the buffer is sorted by timestamp, and attaches metadata such as sampling frequency and device identifiers.

### Step 3:

Terminal transmits a unified request to the server.

Terminal combines the structured question data, the animal attributes, the optional tags, and the recent time-series buffer into a single request payload and sends it to the server via a secure communication protocol.

Input: Structured question object, animal profile, tags, and time-series buffer.

Output: A network request containing a serialized payload (for example, JSON or binary) addressed to a server endpoint.

Terminal serializes the data into a compact representation, compresses the payload if necessary, adds authentication tokens, and sends the payload through the communication interface.

### Step 4:

Server receives and validates the request.

Server accepts the incoming request on an application endpoint, verifies the authentication token, and parses the payload into internal data structures.

Input: Serialized request payload received over the network.

Output: In-memory representations of the user question, animal profile, tags, and time-series data, along with a generated session identifier.

Server deserializes the payload, checks data integrity (for example, presence of mandatory fields and valid ranges), and writes a new session record into persistent storage, linking it to the user and animal identifiers.

### Step 5:

Server preprocesses the natural-language question.

Server cleans and analyzes the question text to extract key terms and intent.

Input: Raw question text string from the user.

Output: A parsed question object including tokenized text, identified keywords (for example, “less active,” “not eating”), and an intent label such as “activity concern” or “appetite concern.”

Server performs tokenization, lowercasing, and punctuation normalization, identifies phrases using lexical patterns, and applies an intent classifier implemented as a machine-learning model to assign an intent label based on the distribution of terms.

### Step 6:

Server preprocesses the time-series sensor data.

Server transforms the raw time-series readings into normalized and segmented data suitable for numerical analysis.

Input: Time-series buffer of sensor readings, each with a timestamp and multiple channels.

Output: A set of fixed-length time windows, each containing normalized feature vectors ready for classification.

Server first resamples the data to a uniform sampling rate, aligns sensor channels in time, applies noise reduction filters such as moving averages or low-pass filters, and then normalizes each channel (for example, to zero mean and unit variance). Server slices the normalized data into contiguous windows of predetermined duration, discarding segments that are too short or incomplete.

### Step 7:

Server computes activity-related features and indices.

Server converts each window into a feature vector and computes overall indices such as activity amount and behavioral tendency.

Input: Segmented, normalized time-series windows.

Output: Window-level feature vectors and global indices including an activity amount index, a behavioral tendency index, and preliminary statistical measures.

Server calculates window features such as mean, variance, energy, and frequency-domain descriptors by applying mathematical functions to each window. Server then aggregates these features across windows to compute summaries like total active time, longest rest duration, and time proportion spent in different movement intensities.

### Step 8:

Server classifies activity patterns and computes an abnormality degree.

Server uses a trained neural network to classify activity type per window and then quantifies deviation from historical patterns.

Input: Window-level feature vectors and stored historical indices for the same animal.

Output: Activity class probabilities per window, aggregated class durations, an activity amount index, a behavioral tendency index, and an abnormality degree value.

Server feeds each feature vector into an activity classifier neural network, obtains class probabilities (for example, walking, running, resting, sleeping), and sums probabilities over time to estimate durations per class. Server compares current indices with historical baselines (for example, using z-scores or other deviation measures) and combines these comparisons into a single abnormality degree according to predefined formulas.

### Step 9:

Server retrieves and integrates history information.

Server accesses stored records for the animal to provide context such as past issues, previous advice, and known conditions.

Input: Animal identifier and session identifier.

Output: A history object including past episodes, previous abnormality degrees, stored advice summaries, and medical notes.

Server runs database queries to retrieve historical records, filters them to a relevant time span, and converts them into a compact summary (for example, recurring patterns of low activity or known food sensitivities). Server then merges this history with the current indices.

### Step 10:

Server analyzes user emotion from text and/or voice.

Server estimates the emotional state of the user from available interaction data.

Input: User speech audio (if available) and/or user text, including the question and recent messages.

Output: Emotion information including an emotion category such as “anxious” or “calm” and one or more confidence scores.

Server extracts acoustic features from audio or textual features from text and applies an emotion classifier, which may be a neural network or other statistical model, to obtain probability values over emotion categories. Server selects the category with highest probability and retains the full probability vector as part of the emotion information.

### Step 11:

Server constructs a unified context object.

Server combines all current and historical information into a structured context for prompt generation.

Input: Parsed question object, animal attribute information, current activity indices and abnormality degree, history object, and emotion information.

Output: A context object containing fields that describe the situation in a machine-readable form.

Server populates a data structure with named fields for each component (question intent, symptom keywords, activity indices, previous abnormality degrees, emotion category, etc.), establishes cross-references with underlying raw data for traceability, and stores this object in working memory.

### Step 12:

Server generates a prompt sentence for the generative AI model.

Server converts the context object into a carefully structured natural-language instruction that guides the generative AI model.

Input: Context object from the previous step and one or more prompt templates.

Output: A prompt sentence (or prompt document) in text form, ready to be tokenized and input to the generative AI model.

Server selects a template based on the intent and abnormality degree (for example, an “activity concern” template), inserts parameter values such as species description, recent activity summary, and a paraphrased user question, and produces a coherent prompt sentence, for example:

“You are a virtual assistant that supports an animal caretaker. Analyze the following pet data and the owner's question. Pet: animal type, approximate age, and known conditions. Recent activity summary: total active time, longest rest period, and abnormality degree. Owner question: ‘My dog has been less active today. Is there a problem?’ Provide concise, concrete advice for the caretaker, including when to consult a veterinarian.”

### Step 13:

Server invokes the generative AI model with the prompt sentence.

Server runs a text generation process based on the generated prompt sentence.

Input: Prompt sentence text and configuration parameters such as temperature, maximum token count, and decoding strategy.

Output: A generated response text sequence containing answer content, recommendations, and explanations.

Server tokenizes the prompt into subword units, maps them to embeddings, runs them through the generative AI model (for example, a transformer network), and iteratively generates output tokens. Server applies configuration parameters that may depend on context (for example, lower temperature when the abnormality degree is high), assembles the tokens back into a text string, and obtains the raw model response.

### Step 14:

Server post-processes the generated response into structured output information.

Server converts the model's raw text into a response that reflects user emotion and is structured for presentation.

Input: Raw generated response text and emotion information.

Output: Output information including advice information, training plan information, and nutrition management information, with tone and style adjusted to the emotion.

Server parses the response into sections (summary, action list, additional notes), checks for safety or unsupported content according to rule sets, and then modifies wording according to emotion information—adding reassuring language if the user is anxious, or providing more detailed explanations if the user appears calm. Server labels segments as advice, training, or nutrition content based on pattern rules and the original prompt specification.

### Step 15:

Server records the interaction and updates prompt generation conditions.

Server stores a complete record of the interaction and adjusts internal parameters for future prompt generation.

Input: Context object, prompt sentence, emotion information, structured output information, and optional user feedback from previous interactions.

Output: Updated record information in storage and updated configuration values influencing future prompt and input conditions.

Server writes a new log entry that links the prompt sentence, the current indices, the emotion category, and the final output information, and aggregates statistics across many such records. Based on observed correlations (for example, which templates yield fewer follow-up questions for a given intent), Server updates template selection rules, parameter defaults such as maximum token counts, and inclusion or exclusion of certain indices in future prompt sentences.

### Step 16:

Server sends the final output information to the terminal.

Server prepares a response message that includes the adjusted answer text and any additional structured metadata, and transmits it to the terminal.

Input: Structured output information prepared in the previous step.

Output: A network response containing the final advice text, optional urgency flags, and metadata such as reference IDs.

Server serializes the output, sets appropriate response codes, and uses the communication interface to send the data to the terminal over the network.

### Step 17:

Terminal displays the advice and auxiliary information to the user.

Terminal receives the server's response and presents the content in an appropriate user interface.

Input: Network response from the server containing answer text and metadata.

Output: Visual or audio output on the terminal that the user can perceive and interact with.

Terminal parses the response, updates the screen with the main advice, highlights urgent alerts using visual cues, and optionally schedules local notifications for follow-up checks.

Terminal may store the advice and session identifier locally so that the user can review the interaction later.

### Step 18:

User reviews the advice and may provide follow-up input.

User reads or listens to the advice, decides on concrete actions for the animal, and may ask follow-up questions.

Input: Displayed advice text and any urgency or category indications.

Output: User actions in the real world (such as changing walking time or feeding pattern) and optional new questions entered into the terminal.

User may tap a button labeled, for example, “Ask a follow-up question,” and type a new question such as “If my dog is still inactive tomorrow, what should I do?”, which becomes new input for another iteration of the processing flow.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

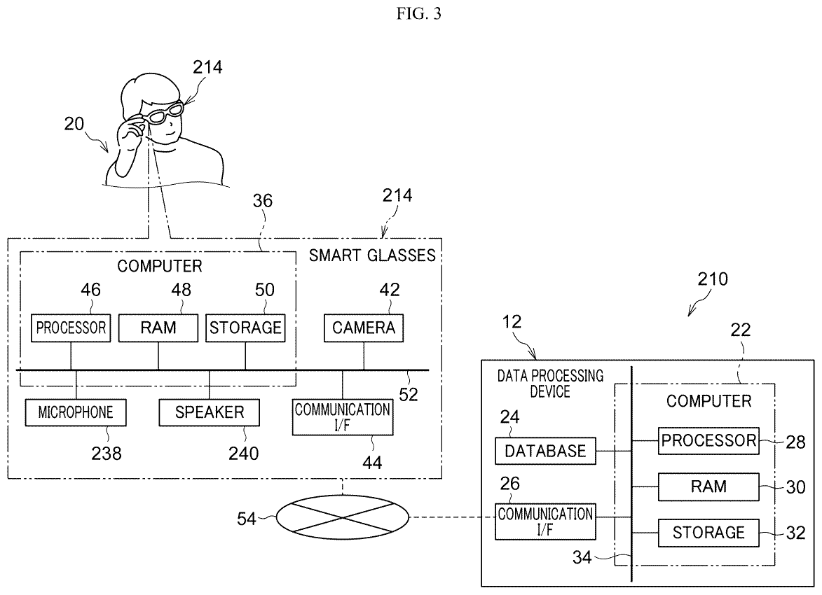

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

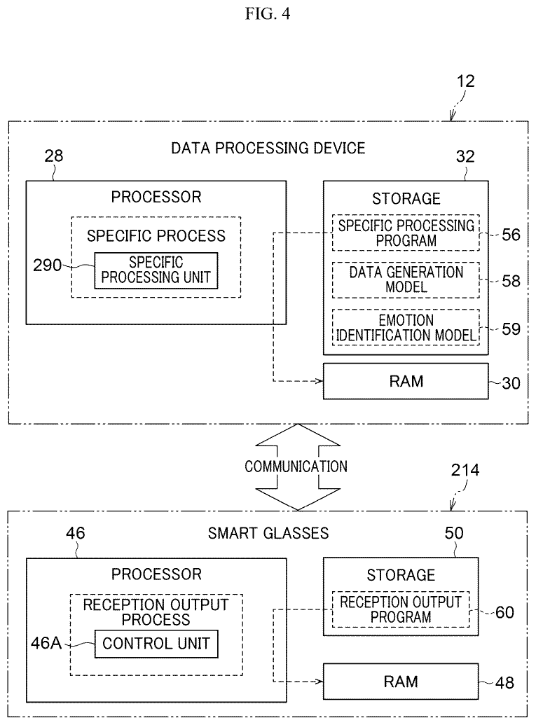

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

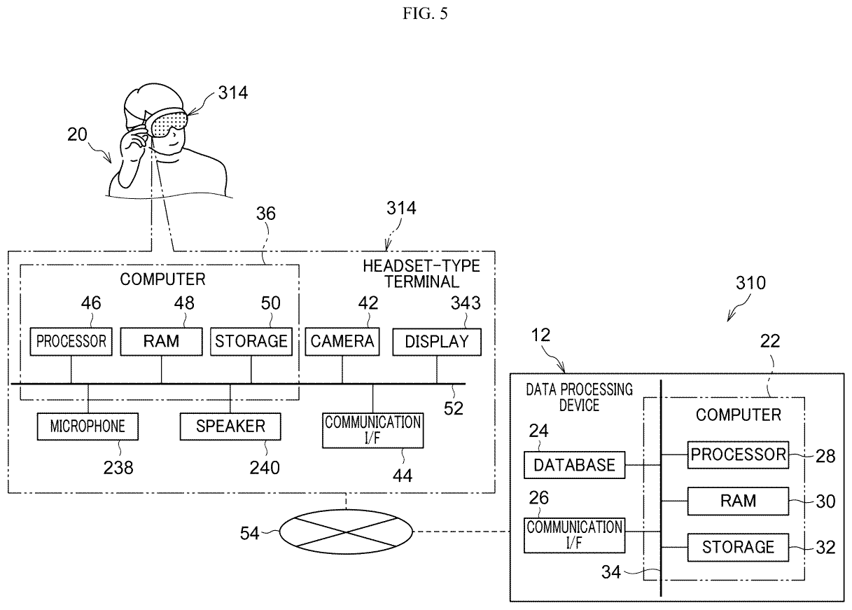

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

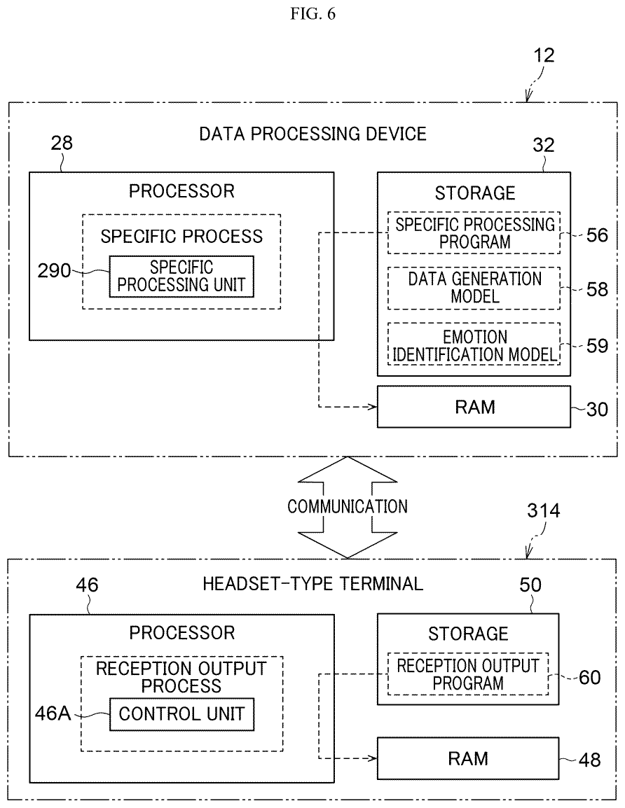

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

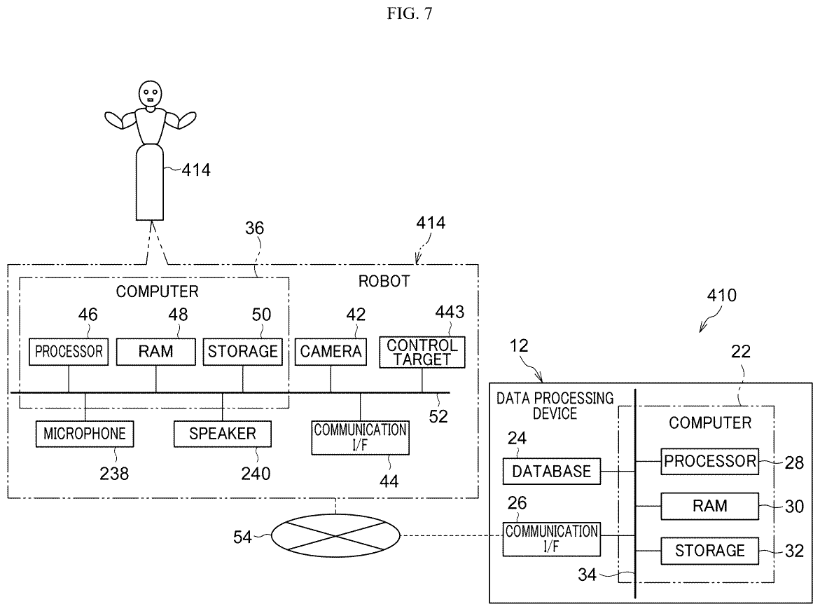

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

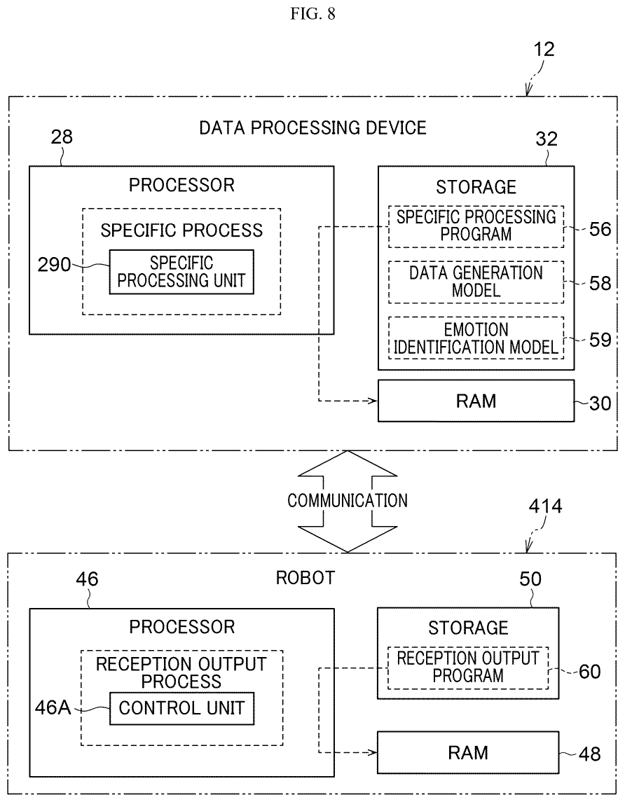

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

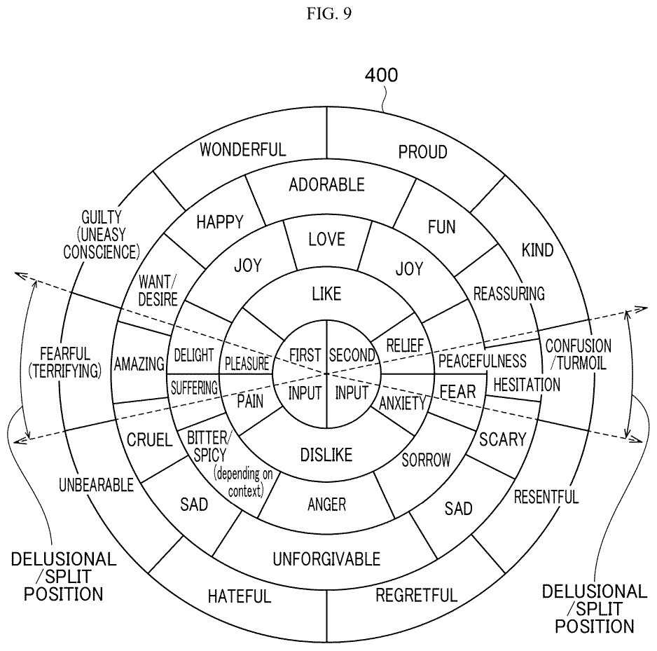

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

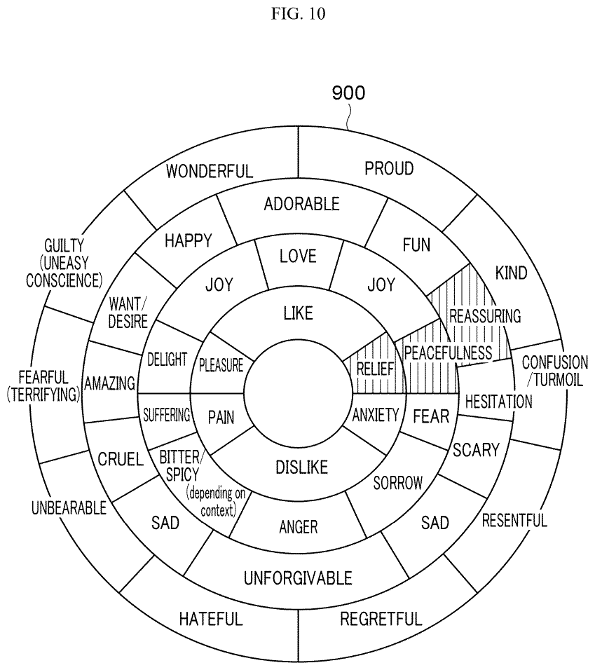

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
receive, from a terminal, a prompt sentence that constitutes inquiry information to be input to a generative information processing model, generate communication information including the prompt sentence, and transmit the communication information to an information processing apparatus,
extract, in the information processing apparatus, the prompt sentence from the communication information, generate analysis information including the prompt sentence and history information related to an animal, input the analysis information to the generative information processing model, and cause the generative information processing model to generate response information based on natural language processing,
access, in the information processing apparatus, a storage device storing health information and behavior information of the animal, and generate personalized response information by adding supplementary information based on the health information and the behavior information to the response information,
transmit, from the information processing apparatus, the personalized response information to the terminal, and in the terminal convert the personalized response information into display information and present the display information via an output device, and
record, as history information, the prompt sentence and the personalized response information transmitted between the terminal and the information processing apparatus, and make the history information available as subsequent input information to the generative information processing model.

### Supplementary 2

The system according to supplementary 1,
wherein the processor is configured to
automatically format inquiry information from a caretaker, in the terminal, as a standardized prompt sentence, combine the prompt sentence with additional information including animal attribute information and caregiving situation information, and transmit the combined information to the information processing apparatus so as to provide structured inquiry information as input to the generative information processing model.

### Supplementary 3

The system according to supplementary 1,
wherein the processor is configured to
generate, in the information processing apparatus, support information including exercise plan information, behavior training plan information, and nutrition management plan information based on the response information output from the generative information processing model and the health information and the behavior information of the animal acquired from the storage device, and transmit the support information as the personalized response information to the terminal.

### Application Example 1

### Supplementary 1

A system comprising a processor, a storage apparatus, a wearable visual information presentation apparatus including a detection apparatus, and a communication apparatus, wherein the processor is configured to
receive information from an animal keeper as a prompt sentence for a generative AI model and initiate a dialogue with the animal keeper,
control the wearable visual information presentation apparatus so that the detection apparatus acquires detection information related to a physical state and a behavioral state of an animal, converts the detection information into digital information, and transmits the digital information to the processor via the communication apparatus,
store the detection information and past historical information in the storage apparatus, perform preprocessing on the detection information and the past historical information by statistical processing or signal processing, and calculate a health state index and a behavioral state index of the animal,
generate, based on the health state index and the behavioral state index, a prompt sentence to be given to the generative AI model, and cause the generative AI model to evaluate a health state of the animal in response to the prompt sentence,
generate, based on an evaluation result from the generative AI model and attribute information of the animal, another prompt sentence for causing the generative AI model to generate in natural language at least one of advice information, training plan information, and nutrition management information for the animal keeper, and obtain the at least one of the advice information, the training plan information, and the nutrition management information from the generative AI model,
transmit the obtained at least one of the advice information, the training plan information, and the nutrition management information to the wearable visual information presentation apparatus via the communication apparatus and cause a display unit of the wearable visual information presentation apparatus to display the at least one of the advice information, the training plan information, and the nutrition management information in a time-series manner to provide real-time care support to the animal keeper, and
analyze an emotional state of the animal keeper by emotion state analysis processing and adjust at least one of the prompt sentence and a response content from the generative AI model according to the emotional state.

### Supplementary 2

The system according to supplementary 1,
wherein the processor is configured to
cause the generative AI model to automatically generate response information by providing the generative AI model with a prompt sentence including inquiry content from the animal keeper and the detection information, modify an expression content or a level of detail of the response information based on the emotion state analysis processing, and present the modified response information via the wearable visual information presentation apparatus.

### Supplementary 3

The system according to supplementary 1,
wherein the processor is configured to
calculate a deviation degree from a reference state for each animal based on the health state index, the behavioral state index, and past health state information and past behavioral state information stored in the storage apparatus, provide the generative AI model with a prompt sentence including information indicating the deviation degree so that the generative AI model automatically adjusts at least one of the advice information, the training plan information, and the nutrition management information for the animal keeper, and further optimize an adjustment content of the at least one of the advice information, the training plan information, and the nutrition management information based on the emotion state analysis processing.

### Example 2

### Supplementary 1

A system comprising a processor,
wherein the processor is configured to
acquire biological information and behavioral information from a detection device and an imaging device attached to an animal, associate the biological information and the behavioral information with time information, and store the associated information,
perform abnormal value removal, interpolation processing, normalization processing, and interval segmentation processing on the biological information and the behavioral information by using statistical processing or signal processing, and generate a time-series information set and an image information set suitable for analysis,
input the time-series information set and the image information set to a generative AI model constructed on a machine learning framework, and estimate behavioral evaluation information including an activity index, a rest-state index, an feeding-behavior index, and an abnormal-behavior index of the animal,
generate management instruction information including an exercise plan, a training plan, a nutrition-management plan, and a medical-consultation recommendation by configuring a prompt sentence using the behavioral evaluation information, attribute information of an individual animal, and target activity-level information, and by inputting the prompt sentence to the generative AI model to generate the management instruction information expressed in a natural language,
perform emotion-analysis processing to estimate an emotional state from interactive input information and image information, and adjust an expression format of the prompt sentence or a presentation content of the management instruction information in accordance with the emotional state,
transmit the management instruction information and the adjusted presentation content to an information terminal via a communication network, and cause the information terminal to present notification information, and
store evaluation information or additional input information provided by a user via the information terminal with respect to the management instruction information, and update a configuration of the prompt sentence or calculation conditions of the behavioral evaluation information based on the evaluation information or the additional input information.

### Supplementary 2

The system according to supplementary 1,
wherein the processor is configured to
aggregate a history of the behavioral evaluation information and the management instruction information for each predetermined period, configure a prompt sentence for generating summary information for the predetermined period, cause the generative AI model to generate a report sentence including the summary information based on the prompt sentence, and provide the report sentence to the information terminal.

### Supplementary 3

The system according to supplementary 1,
wherein the processor is configured to
configure a prompt sentence for automatically generating response content based on question content included in the interactive input information and based on at least one of the biological information, the behavioral information, and the behavioral evaluation information, cause the generative AI model to generate the response content based on the prompt sentence, adjust at least one of tone, level of detail, and presentation order of the response content in accordance with the emotional state, and provide the adjusted response content to the information terminal.

### Application Example 2

### Supplementary 1

A system comprising a processor,
wherein the processor is configured to
analyze natural language input information acquired from an animal caretaker, and generate a prompt sentence to be provided to a generative artificial intelligence model based on the input information, attribute information related to an animal, and time-series behavioral information related to the animal, and instruct the generative artificial intelligence model to start interactive processing by using the prompt sentence,
record observation information including biological information of the animal and behavioral information of the animal, acquired from a detection unit, as time-series data, perform preprocessing on the time-series data including at least noise reduction, normalization, and temporal segmentation, and generate an analysis result indicating at least an activity amount, a behavioral tendency, and an abnormality degree by using the preprocessed time-series data, construct a prompt sentence to be input to the generative artificial intelligence model based on the analysis result, history information related to the animal, and the natural language input information, and convert response information acquired from the generative artificial intelligence model into output information including at least advice information, training plan information, and nutrition management information to be presented to the animal caretaker, analyze voice information or character information of a user to calculate emotion information indicating an emotional state of the user, and adjust at least a content of expression and a presentation manner of the output information based on the emotion information, and
store the output information and the emotion information in association with each other as record information, and update at least a generation condition for the prompt sentence or an input condition for the generative artificial intelligence model based on the stored record information.

### Supplementary 2

The system according to supplementary 1,
wherein the processor is configured to
generate the prompt sentence based on context information including a question sentence acquired from the animal caretaker, the time-series data, and the history information, and automatically output the response information acquired from the generative artificial intelligence model as answer information to which expression adjustment based on the emotion information has been applied.

### Supplementary 3

The system according to supplementary 1,
wherein the processor is configured to
automatically generate the advice information, the training plan information, and the nutrition management information for the animal caretaker based on at least an activity index and a health state index calculated from the analysis result, and further adjust at least a content and a presentation timing of the advice information, the training plan information, and the nutrition management information based on the emotion information.

What is claimed is:

1. A system comprising:
circuitry configured to:
receive, via a communication interface coupled to a packet-switched network, a prompt sentence constituting inquiry information from a terminal device, generate communication information including the prompt sentence, and transmit the communication information to a generative AI model;
extract the prompt sentence from the communication information, generate analysis result information by applying the generative AI model to the prompt sentence, and generate response information based on the analysis result information; and
control acquisition of sensor data representing a condition and behavioral patterns of a monitored subject from at least one sensor or imaging device via the communication interface, perform preprocessing on the sensor data, and incorporate the preprocessed sensor data into a subsequent prompt sentence for input to the generative AI model.

2. The system according to claim 1, wherein the circuitry is configured to receive the prompt sentence from the terminal device via the communication interface, generate communication information incorporating the prompt sentence, and initiate a dialog session with the terminal device based on the received prompt sentence.

3. The system according to claim 2, wherein the circuitry is configured to extract the prompt sentence from the communication information received from the terminal device, input the prompt sentence to the generative AI model to generate analysis result information, and generate response information from the analysis result information for transmission to the terminal device.

4. The system according to claim 3, wherein the circuitry is configured to transmit the response information to the terminal device via the communication interface, and receive a follow-up prompt sentence from the terminal device based on the transmitted response information.

5. The system according to claim 1, wherein the circuitry is configured to control acquisition of sensor data from at least one of a sensor and an imaging device via the communication interface, perform preprocessing on the sensor data including normalization and removal of noise, and generate structured monitoring data from the preprocessed sensor data.

6. The system according to claim 5, wherein the circuitry is configured to incorporate the structured monitoring data into a prompt sentence for the generative AI model, input the prompt sentence to the generative AI model to generate an analysis result based on the monitoring data, and transmit the analysis result to the terminal device.

7. The system according to claim 1, wherein the circuitry is configured to recognize an emotional state of a user associated with the terminal device by applying an emotion analysis engine to data received from the terminal device via the communication interface, and adjust the response information based on the recognized emotional state.

8. The system according to claim 7, wherein the circuitry is configured to adjust at least one of a content selection, a presentation format, and a detail level of the response information based on the recognized emotional state of the user.

9. The system according to claim 1, wherein the circuitry is configured to combine the prompt sentence from the terminal device with the structured monitoring data in a combined prompt sentence, input the combined prompt sentence to the generative AI model, and obtain a combined analysis result that integrates inquiry information and monitoring data.

10. The system according to claim 9, wherein the circuitry is configured to detect a condition anomaly in the structured monitoring data that exceeds a threshold, generate an alert prompt incorporating the anomaly details, and input the alert prompt to the generative AI model to obtain recommended actions.

11. The system according to claim 10, wherein the circuitry is configured to transmit the recommended actions to the terminal device via the communication interface, and store the anomaly details and recommended actions in a storage device in association with a subject identifier.

12. The system according to claim 1, wherein the circuitry is configured to store dialog history between the terminal device and the generative AI model in a storage device, and incorporate the dialog history into subsequent prompt sentences to maintain conversational context.

13. The system according to claim 12, wherein the circuitry is configured to detect a topic transition in the dialog history based on the content of successive prompt sentences received from the terminal device, update the dialog context data to reflect the transition, and adjust the subsequent prompt sentence accordingly.

14. The system according to claim 1, wherein the circuitry is configured to collect behavioral pattern data from the imaging device via the communication interface, apply a pattern recognition algorithm to the behavioral pattern data, and generate a behavioral summary for incorporation into a prompt sentence for the generative AI model.

15. The system according to claim 14, wherein the circuitry is configured to detect anomalies in the behavioral pattern data that exceed a significance threshold, and automatically generate a prompt sentence for the generative AI model incorporating the anomaly details to obtain recommended responses.

16. The system according to claim 1, wherein the circuitry is configured to receive feedback from the terminal device on the relevance and usefulness of the transmitted response information, store the feedback in the storage device, and incorporate the feedback into subsequent prompt sentences to improve response accuracy.

17. The system according to claim 16, wherein the circuitry is configured to update parameters of the generative AI model based on accumulated feedback stored in the storage device, and use the updated model for subsequent prompt processing.

18. A system comprising:
circuitry configured to:
receive, via a communication interface coupled to a packet-switched network, a prompt sentence from a terminal device, generate communication information including the prompt sentence, and input the prompt sentence to a generative AI model to generate response information;
control acquisition of sensor data from at least one sensor or imaging device via the communication interface, perform preprocessing on the sensor data, and incorporate the preprocessed sensor data into a subsequent prompt sentence for the generative AI model;
recognize an emotional state of a user based on data received from the terminal device via the communication interface, and adjust the response information based on the recognized emotional state; and
transmit the adjusted response information to the terminal device via the communication interface, and store dialog history in a storage device for incorporation into subsequent prompt sentences.

19. The system according to claim 18, wherein the circuitry is configured to detect a condition anomaly in the preprocessed sensor data that exceeds a threshold, generate an alert prompt incorporating the anomaly details, input the alert prompt to the generative AI model to obtain recommended actions, and transmit the recommended actions to the terminal device.

20. A method comprising:
receiving, via a communication interface coupled to a packet-switched network, a prompt sentence from a terminal device, generating communication information including the prompt sentence, and transmitting the communication information to a generative AI model;
extracting the prompt sentence from the communication information, generating analysis result information by applying the generative AI model to the prompt sentence, and generating response information based on the analysis result information; and
controlling acquisition of sensor data from at least one sensor or imaging device via the communication interface, performing preprocessing on the sensor data, and incorporating the preprocessed sensor data into a subsequent prompt sentence for input to the generative AI model.
